(19) United States
(12) Patent Application Publication
(10) Pub. No.: US 2006/0004409 A1
(43) Pub. Date: Jan. 5, 2006

(54) Devices for locking and/or cutting a suture

(76) Inventors: Rudolph H. Nobis, Mason, OH (US); Omar J. Vakharia, Mason, OH (US); John A. Faux, Cincinnati, OH (US); Christopher Paul Swain, London (GB); Charles A. Mosse, London (GB); Annette Dora Anna Fritscher-Ravens, Bruchhausen-Vilsen (DE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/127,952

(22) Filed: May 12, 2005

Related U.S. Application Data
(60) Provisional application No. 60/571,000, filed on May 14, 2004.

(51) Int. Cl. A61B 17/04

(52) U.S. Cl. 606/232

(57) ABSTRACT

Endoscopic suturing devices are provided for suture locking and/or cutting, the devices being small enough to pass through the working channel of various endoscopic and ultrasound devices. One embodiment of the invention provides a device and method for a physician in a medical procedure to automatically lock and cut a suture in one motion and without the need for additional cutting instrumentation, rather than perform separate locking and cutting actions.

### CROSS-REFERENCES TO RELATED APPLICATIONS

This application claims priority to and incorporates by reference U.S. Provisional Patent Application No. 60/571,000 filed May 14, 2004 entitled “Suture locking and cutting mechanisms that are suitably small enough to pass through the working channel of an endoscope”.

### FIELD OF THE INVENTION

This invention relates to endoscopic suturing devices. More particularly, this invention relates to suture locking and cutting mechanisms that are small enough to pass through the working channel of various endoscopic and ultrasound devices.

### BACKGROUND

Application of sutures in the gastrointestinal tract is required for several different types of medical procedures, for example, for transoral endoscopic valvuloplasty for gastroesophageal reflux disease (GERD), gastroplasty, fundoplication, anterior gastropexy, posterior gastropexy, suturing esophageal perforations, or closure of the esophageal side of the tracheo-esophageal fistula. Traditionally, these procedures are performed by physicians, such as gastroenterologists or surgeons, either by laparoscopy or open surgical techniques. Such procedures are invasive, as laparoscopy requires that small access incision(s) be made in the body of the patient, through which a laparoscope and other surgical enabling tools are provided, while open surgical techniques are traditionally invasive and can have complications and cause long patient recovery periods.

The solution to these problems is to perform these medical procedures through the gastroesophageal tract via the mouth or other naturally occurring orifice. Already available flexible endoscopes, commonly called gastroscopes, can be provided through the gastroesophageal tract and enable illumination and visualization of tissue along the gastroesophageal tract on a video display for diagnostic purposes. These flexible endoscopes also provide an instrumentation means for applying sutures in tissue, such as in the wall of the stomach. What is needed are improved methods of providing a totally transoral surgical procedure, such as a posterior gastropexy procedure, and thereby avoid more invasive laparoscopic procedures.

New endoscopic suturing methods performed through the gastroesophageal tract as an alternative to the invasive laparoscopic method of, for example, a posterior gastropexy procedure, are currently being developed. For example, suturing methods under the control of endoscopic ultrasound (EUS) are being evaluated. EUS is a procedure that combines endoscopy and ultrasound. In particular, a Mar. 14, 2003 publication authored by Fritscher-Ravens, Mosse, Mukherjee, Yazaki, Park, Mills, and Swain, entitled, “Transgastric gastropexy and hiatal hernia repair for GERD under EUS control: a porcine model,” (American Society for Gastrointestinal Endoscopy) describes how endoluminal operations for gastroesophageal reflux are currently limited by the inability of the surgeon to visualize and manipulate structures outside the wall of the gut. The publication describes a way to define the EUS anatomy of structures outside the gut that influence reflux, to place stitches in the median arcuate ligament, to perform posterior gastropexy, and to test the feasibility of crural repair, under EUS control, in pigs. More specifically, by using a linear-array EUS, the median arcuate ligament and part of the right crus were identified and punctured with a needle, which served as a carrier for a tag and suture. These were anchored into the muscle. An endoscopic sewing device was used, which allowed stitches to be placed through a 2.8-mm accessory channel to any predetermined depth.

The publication also describes new methods of knot tying and suture cutting through the 2.8-mm channel of the EUS. More specifically, stitches were placed through the gastric wall into the median arcuate ligament, and one stitch was placed just beyond the wall of the lower esophageal sphincter. The stitches were tied together and locked against the gastric wall, and the surplus length of suture material was then cut and removed. While this publication describes a suitable transgastric gastropexy and hiatal hernia repair procedure, further improvements in methodology and equipment to perform such procedures would be beneficial. For example, the publication describes a process for locking and cutting the suture from inside the stomach. However, the suture requires that a separate suture cutting step, along with its associated cutting instrumentation, be available via the working channel of the endoscope. This may result in multiple passes of instrumentation back and forth through the working channel of the endoscope. What is needed is a way to both lock and cut a suture automatically with a single device and thereby simplify the medical procedure, such as a posterior gastropexy procedure.

It is therefore an object of an aspect of the invention to provide improved methods of performing a totally transoral surgical procedure, such as a posterior gastropexy procedure, and thereby avoid more-invasive laparoscopic procedures.

Additionally, the locking mechanism described in the publication is too large to pass through the working channel of an endoscope and, thus, it must be inserted into the patient separately from the endoscope, which again adds complexity to the medical procedure. What is needed are suture locking and cutting mechanisms that are small enough to pass through the working channel of various endoscopic and ultrasound devices (typical working channel diameter is 2.8-3.4 mm).

Various forms of suture-locking device are described in U.S. Pat. No. 4,235,238 (Ogiu et al). In particular, this US patent describes various forms of suture-locking device which employ some form of suture-finishing stop, which, it is asserted, can be used to clamp the suture. However, all the designs described appear to be inherently unreliable (they are likely either not to clamp the suture in the first place, or, if they have done so, to work loose subsequently), or they lack flexibility in terms of how they can used (the lock can be made progressively tighter by the endoscopist, but the process can never be reversed if the endoscopist has made it too tight). There is thus a need for a suture-locking device which can overcome these problems.

A suture cutting device is described in GB-A-2247841. This employs a cutting tube which is slideable over an elongated rod, the rod having a pair of eyelets through which the suture material to be cut passes. However, the device is not described as being useable via a flexible endoscope, and appears in fact only to be useable during rigid endoscopy.

Another suture cutting device is described in Japanese Utility Model Application No. 158729/1978. However, this attempts to hold the suture during cutting by means of a pair of open-ended slots in an outer member, and a corresponding slot in an inner member. This is unlikely to hold the suture securely under many circumstances, thereby rendering it unsatisfactory for surgical use.

WO95/25470 describes a suture cutting device which is for use in conjunction with a flexible endoscope. However, this achieves its cutting action by having the suture passing through slots in an inner member, and then around the outside of the endoscope. Also, the technique requires the suture to be held under tension during the cutting operation. This combination of features means that the cutting operation may not be as reliable as is desired.

It is an object of other aspects of this invention to provide a single mechanism for automatically locking and/or cutting a suture and thereby simplifying medical procedures, such as, but not limited to, a posterior gastropexy procedure.

It is yet another object of this invention to provide suture locking and cutting mechanisms that are small enough to pass through the working channel of various endoscopic and ultrasound devices.

### SUMMARY OF THE INVENTION

Certain embodiments of the present invention are directed to providing improved methods of performing a totally transoral surgical procedure, such as a posterior gastropexy procedure, and thereby avoiding more-invasive laparoscopic procedures. One embodiment of the present invention provides a device and method that allows a physician in a medical procedure to automatically lock and cut a suture in one motion and without the need for additional cutting instrumentation, rather than perform separate locking and cutting actions.

In one embodiment of the invention, a suture lock assembly in combination with a lock actuating device is provided. The lock comprises an extension spring arranged between two endcaps, wherein one or more sutures are locked within the coils thereof. Extending the extension spring allows for one or more sutures to be threaded therethrough and, by relaxing the extension spring, provides a clamping action upon the sutures and a tortuous path within the coils. The lock actuating device provides a cutting mechanism. Furthermore, both the suture lock assembly, in combination with a lock actuating device, are suitably small enough to pass through the working channel of various endoscopic and ultrasound devices.

In another embodiment of the invention, a suture lock assembly is provided that forms a hollow body, within which a clamp device is engaged and through which one or more sutures is threaded. Depending upon the slidable position of the clamp device within the body, the suture within the clamp device is engaged to clamp the suture permanently. The suture lock assembly of this embodiment is likewise suitably small enough to pass through the working channel of various endoscopic and ultrasound devices.

In yet another embodiment of the invention there is provided a suture-locking device which comprises an outer tubular member, and an inner tubular member which has a distal portion of a first cross-section and a proximal portion of a second cross-section, the said first portion having an aperture formed therethrough and sized to allow a suture to pass therethrough, the device having a non-locking state in which the said second portion is at least partly received in the outer tubular member, and the said aperture is so located that the suture can pass freely through it, and a locking state in which the said first portion is located at least partially within the outer tubular member, and the suture is locked between the inner and outer tubular members. Preferably, the first and second portions of the inner tubular member are connected to one another by an intermediate portion. More preferably, the first and second portions are at least substantially cylindrical, and the intermediate portion is a tapered portion which integrally connects the first and second portions.

In another aspect of the invention, which may be combined with the immediately preceding embodiment, a suture-locking device is provided which comprises a pair of locking members movable with respect to one another from a non-locking position to a locking position, and pulling means for effecting movement of one of the tubular members relative to the other, the pulling means being connected to the said one member by a connection which is sufficiently strong to enable a force to be applied thereto to effect that relative movement, but which is breakable under a higher force to allow the pulling means to be detached from the tubular members after locking.

In a further embodiment of the invention there is provided a device for cutting a surgical suture, the device comprising a tubular member having a longitudinal axis and a tubular wall with a pair of apertures extending therethrough, the apertures being sized and arranged to permit a surgical suture to pass into the tubular member through one of the pair of apertures and out of the tubular member through the other, a cutting member received within the tubular member, and means for causing longitudinal movement of the cutting member and tubular member with respect to one another in a direction to cause the cutting member to pass at least one of the pair of apertures to sever the suture passing therethrough. Preferably the pair of apertures are preferably longitudinally spaced from one another. They are also preferably offset with respect to one another about the longitudinal axis of the tubular member, and more preferably they are offset from one another by 180 degrees, or approximately 180 degrees.

In still another embodiment of the invention there is provided a device for locking and cutting a suture, which comprises a first member having an aperture sized to allow a suture to pass through, the first member having a distal end and a proximal end, a second member with respect to which the first member is movably mounted, and means releasably connected to the first member for pulling it in a proximal direction from a first position in which the suture is free to pass through the said aperture, via a second position in which the suture is clamped between the first and second members, to a third position in which the suture is cut by cooperation between the first and second members.

In a preferred aspect of the immediately preceding embodiment, the second member is generally cylindrical, and the first member is slidable within the first member. In the first position only the proximal end portion of the first member is received within the second member. In the second position the first member is received within the second member to a greater extent. In the third position the first member is at least substantially received within the first position. The releasable connection between the pulling means and the first member is arranged to separate when a force is applied to the pulling member sufficiently in excess of that required to move the first member into the third position.

### BRIEF DESCRIPTION OF THE DRAWINGS

While the novel features of the invention are set forth with particularity in the appended claims, the invention, in all its embodiments, may be more fully understood with reference to the following description and accompanying drawings.

FIG. 1A illustrates a perspective view of a suture lock assembly in accordance with a first embodiment of the invention;

FIG. 1B illustrates a cross-sectional view of the suture lock assembly in accordance with a first embodiment of the invention;

FIG. 2 illustrates a side view of an exemplary lock actuating device;

FIGS. 3A and 3B illustrate a side view and a top view, respectively, of the distal end of the lock actuating device with the suture lock assembly of the first embodiment engaged therein in the default state;

FIGS. 4A and 4B illustrate a side view and a top view, respectively, of the distal end of the lock actuating device with the suture lock assembly of the first embodiment engaged therein in the lock state;

FIGS. 5A and 5B illustrate a side view and a top view, respectively, of the distal end of the lock actuating device with the suture lock assembly of the first embodiment engaged therein in the cut state;

FIG. 6 illustrates a side view of the suture lock assembly of the first embodiment engaged therein in the release state;

FIG. 7 illustrates a flow diagram of an example method of using the suture lock assembly of the first embodiment in combination with the lock actuating system;

FIG. 8 illustrates a perspective view of a suture lock assembly in accordance with a second embodiment of the invention.

FIG. 9 illustrates a cross-sectional view of the suture lock assembly of the second embodiment in the unlocked state.

FIG. 10 illustrates a cross-sectional view of the suture lock assembly of the second embodiment in the locked state.

FIGS. 11A and 11B illustrate cross section views of an alternative locking device with a one-way flap inside a tubular segment, in a loading state, and a locked state, respectively.

FIGS. 12A and 12B show a cross section view of alternative one-piece clip in a default state, and a cross section view of the one-piece clip in a locked state, respectively.

FIG. 13 shows the head of an embodiment of locking device, on a much enlarged scale, with a suture therein ready to be locked;

FIG. 14 is a disassembled view of the head of FIG. 13, showing the individual components;

FIG. 15 shows the head of FIGS. 13 and 14 and Bowden cable and handle with which it is associated to form a complete locking device;

FIGS. 16a to 16g show successive steps in the operation of the locking device of FIGS. 13 to 15;

FIG. 17 is a longitudinal section, on a much enlarged scale, of an embodiment of cutter head;

FIG. 17a is a similar section of a modified version of the device of FIG. 17;

FIG. 18 shows the complete cutting device, including the cutter head of FIGS. 17 and 17a;

FIG. 19 shows the flexible endoscope with which the device of FIGS. 17 to 18 is to be used;

FIGS. 20a through 20f show successive stages in a cutting procedure using the cutter of FIGS. 17 through 19;

FIG. 21 shows the components of a combined locking and cutting device in disassembled form;

FIG. 22 shows the components of FIG. 21 in assembled form;

FIG. 23 shows the device of FIGS. 21 and 22 in the process of being introduced through the working channel of a flexible endoscope;

FIGS. 24a and 24b show the device of FIGS. 21 and 22 positioned adjacent an area of tissue, with FIG. 24b being on a larger scale than FIG. 24a; and

FIGS. 25a through 25d show successive stages in the operation of the device of FIGS. 21 and 22.

### DETAILED DESCRIPTION OF THE INVENTION

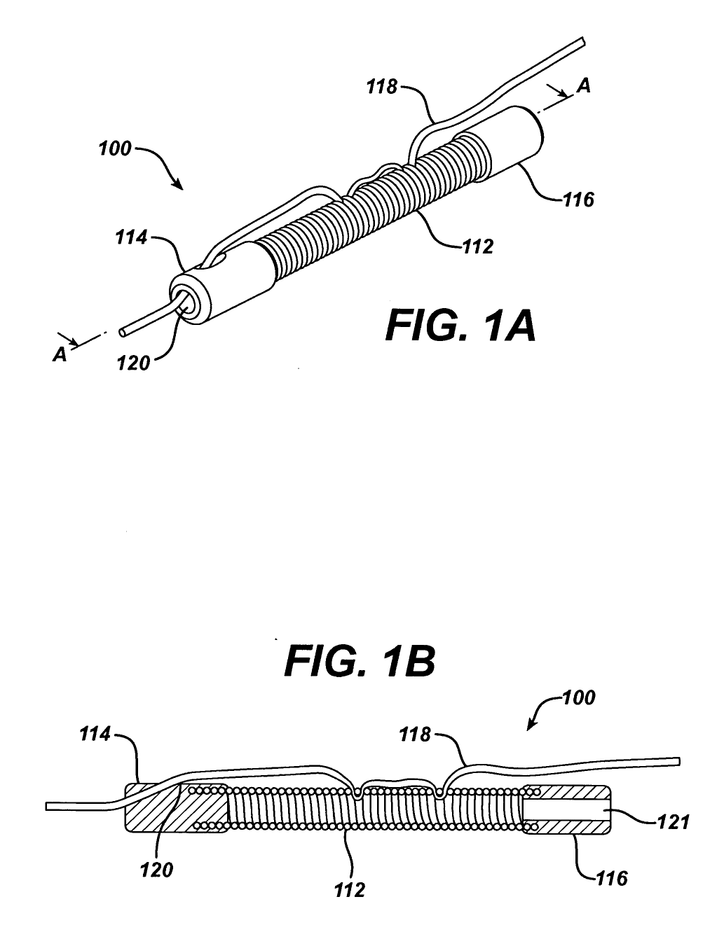

FIG. 1A illustrates a perspective view of a suture lock assembly 100 in accordance with a first embodiment of the invention. Suture lock assembly 100 includes an extension spring 112 arranged between a distal endcap 114 and a proximal endcap 116. The endcaps preferably have an outer diameter of about 0.07 inch. Extension spring 112 is formed of any nontoxic, noncorrosive metal, such as stainless steel, and distal endcap 114 and proximal endcap 116 are formed of, for example, molded plastic or stainless steel. Also shown in FIG. 1 is a suture 118 threaded first through a hole 120 in distal endcap 114 and then through multiple coils of extension spring 112, wherein suture 118 is clamped because of the pressure of the coils and the tortuous path within the coils. Suture lock assembly 100 is not limited to a single suture 118 installed therein; a plurality of sutures 118 may be engaged within a single suture lock assembly 100.

FIG. 1B illustrates a cross-sectional view of suture lock assembly 100 taken along line AA of FIG. 1A. This view shows that proximal endcap 116 further includes a hollow channel 121 that runs through its center. Furthermore, hole 120 in distal endcap 114 is angled from the center of an outer end of distal endcap 114 toward the sidewall of distal endcap 114, preferably at an angle of about 45° to the central axis which thereby allows suture 118 to exit distal endcap 114 external to extension spring 112. Distal endcap 114 and proximal endcap 116 may be insert-molded onto extension spring 112 or use other methods or procedures of providing a smooth, trauma free extension of spring coils.

In operation, suture 118 is threaded first through hole 120 in distal endcap 114; extension spring 112 is then extended and suture 118 is threaded through multiple coils of extension spring 112; extension spring 112 is then relaxed, which thereby applies a tortuous path in addition to a clamping or locking action upon suture 118 between the coils thereof. The overall diameter of suture lock assembly 100 is suitably small enough to allow it to pass through the working channel of various endoscopic and ultrasound devices, which is typically between 2.8 and 3.4 mm in diameter. See Table 1 for example dimensions of suture lock assembly 100.

TABLE 1
Example dimensions of suture lock assembly 100

| | Example Dimension | |
|---|---|---|
| Suture lock assembly 100 overall length | 0.70 | in |
| Extension spring 112 outside diameter | 0.060 | in |
| Extension spring 112 inside diameter | 0.040 | in |
| Distal endcap 114 outside diameter | 0.07 | in |
| Distal endcap 114 length | 0.15 | in |
| Proximal endcap 116 outside diameter | 0.07 | in |
| Proximal endcap 116 length | 0.125 | in |
| Hollow channel 121 diameter | 0.04 | in |
| Hole 120 diameter | 0.04 | in |
| Hole 120 angle | 45 | degrees |

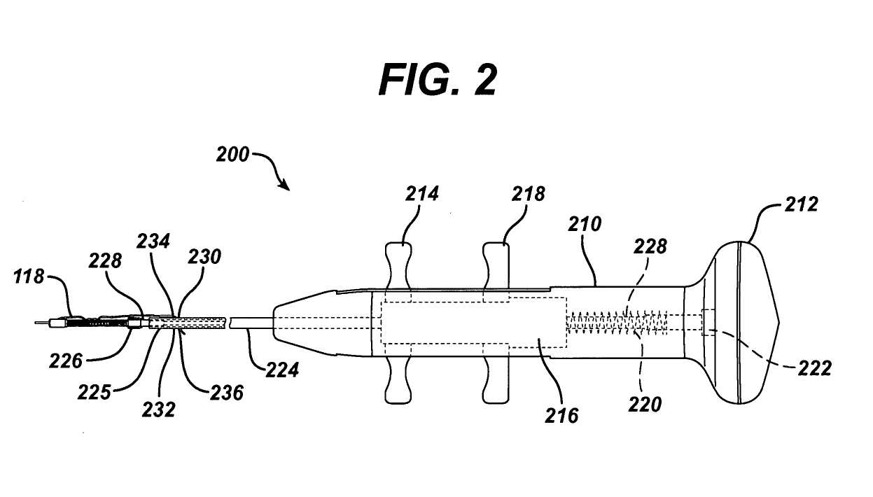

FIG. 2 illustrates a side view of a lock actuating device 200, which is exemplary only and representative of any suitable actuating device for use with suture lock assembly 100. In this example, lock actuating device 200 includes a body 210 that has a knob 212 arranged at its proximal end for grasping by the user. Mechanically coupled to body 210 is a retract handle 214, which has a retract handle body 216 and a retention handle 218 that is slidably arranged within retract handle body 216. Furthermore, a compression spring 220 is mechanically coupled between a spring retainer 222, which is coupled to knob 212, and the proximal end of retract handle body 216. Mechanically coupled to the distal end of retract handle body 216 is a hollow retractable sleeve 224, within which is first arranged a hollow retention sleeve 225, which has a retention jaw 226 at its distal end. Furthermore, arranged within retention sleeve 225 is an actuating shaft 228. FIG. 2 also shows that arranged within the distal end of retractable sleeve 224 is a first slot 230 that is aligned opposite a second slot 232. Also, arranged within the distal end of retention sleeve 225 is a hole 234 that is aligned opposite a slot 236.

Actuating shaft 228 of a fixed length is mechanically coupled at one end to the distal end of spring retainer 222 while passing through spring retainer 222. Actuating shaft 228 passes through a hollow channel within retract handle body 216, then passes through the hollow channel of retention jaw 226 within retractable sleeve 224. The tip of actuating shaft 228 extends through an opening at the distal end of retention jaw 226 within retractable sleeve 224. Using retract handle 214 and retention handle 218, retractable sleeve 224 and retention sleeve 225 are slidable along the length of actuating shaft 228. As a result, the relative axial position of retractable sleeve 224, retention jaw 226, and actuating shaft 228 may vary one to another under user control. Lock actuating device 200 may include well-known mechanical methods and elements (not shown) for holding retractable sleeve 224 and retention jaw 226 at various positional states.

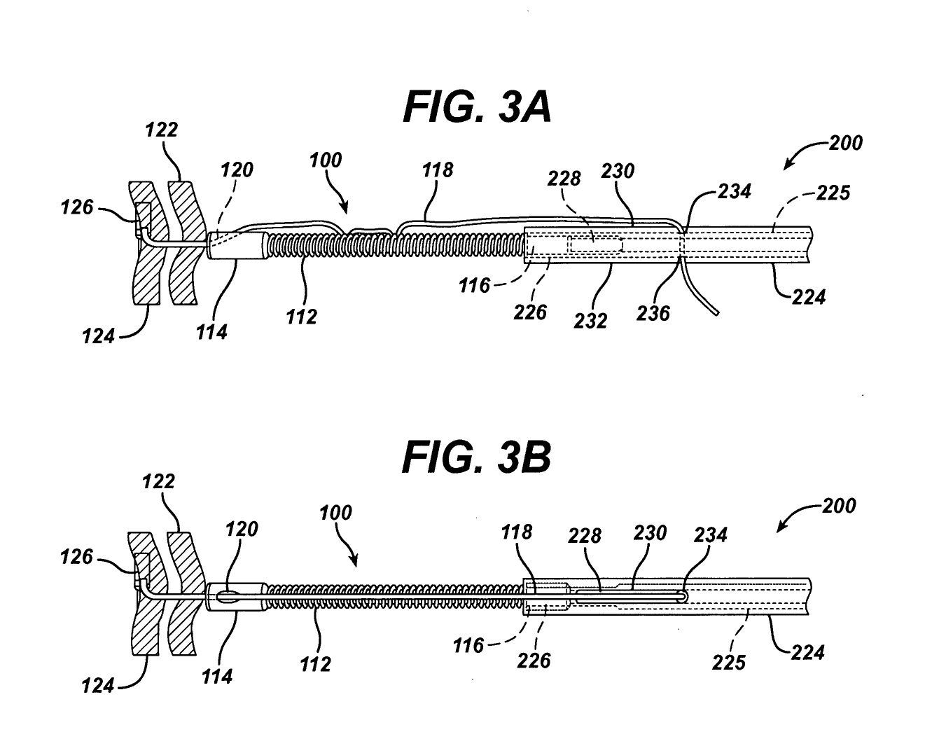

The operation of suture lock assembly 100 in combination with lock actuating device 200 for automatically locking and cutting a suture includes a sequential transition from a default state (i.e., undeployed state) to a lock state, a cut state and, finally, a release state (i.e., deployed state), as described in reference to FIGS. 3A, 3B, 4A, 4B, 5A, 5B, 6, and 7. Additionally, FIGS. 3A, 3B, 4A, 4B, 5A, 5B, and 6 show suture lock assembly 100 in use and, therefore, it includes suture 118, which runs through the center of suture lock assembly 100 and approximates a first tissue 122 and a second tissue 124. Suture 118 is anchored to second tissue 124 with a T-tag 126, which is a well-known medical device for anchoring a suture into body tissue.

FIGS. 3A and 3B illustrate a side view and a top view, respectively, of the distal end of lock actuating device 200 with suture lock assembly 100 engaged therein in the default state, which is described as follows.

Default State:

In the default or undeployed state, extension spring 112 is extended suitably to allow suture 118 to slide freely through its coils. This is accomplished by the physician's passing actuating shaft 228 through hollow channel 121 of proximal endcap 116, then through the center of extension spring 112, until the tip of actuating shaft 228 abuts the inner surface of distal endcap 114. By using retention handle 218, which is attached to the proximal end of retention sleeve 225, the physician extends retention jaw 226 to allow it to grip proximal endcap 116 and then pull proximal endcap 116 into the tip of retractable sleeve 224, as shown in FIGS. 3A and 3B, which thereby extends extension spring 112, relative to the tip of actuating shaft 228. The distance between the tip of actuating shaft 228 and the tip of retractable sleeve 224 is predetermined to suitably extend extension spring 112. Additionally, suture 118 is threaded first through hole 120 in distal endcap 114, then within the extended coils of extension spring 112 is wrapped multiple times around actuating shaft 228, through hole 234 of retention sleeve 225, through first slot 230 of retractable sleeve 224, passes around actuating shaft 228, through slot 236 of retention sleeve 225 and, finally, through second slot 232 of retractable sleeve 224.

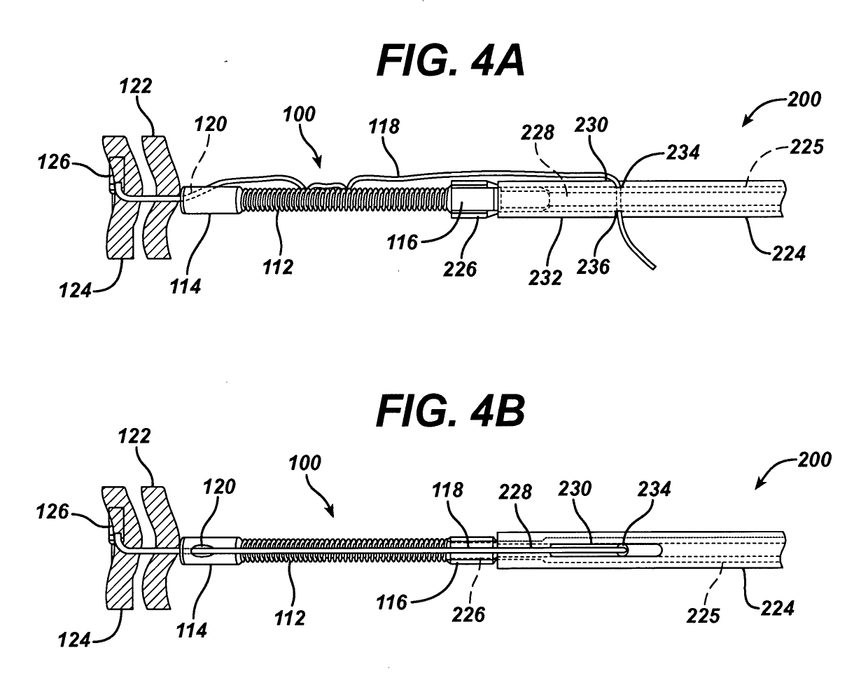

FIGS. 4A and 4B illustrate a side view and a top view, respectively, of the distal end of lock actuating device 200 with suture lock assembly 100 engaged therein in the lock state, which is described as follows.

Lock State:

In the lock state, extension spring 112 is relaxed, which allows its coils to clamp against suture 118 and thereby prevent suture 118 from sliding freely between the coils of extension spring 112. By using retention handle 218, which is attached to the proximal end of retention sleeve 225, the physician extends retention jaw 226 while gripping proximal endcap 116 in a direction toward distal endcap 114 and while maintaining the relative distance between the tip of actuating shaft 228 and the tip of retractable sleeve 224, as set in the default state. Although the relative position of hole 234 and slot 236 to first slot 230 and second slot 232, respectively, is changed, suture 118 is intact and passing freely through hole 234 of retention sleeve 225, through first slot 230 of retractable sleeve 224, passes around actuating shaft 228, through slot 236 of retention sleeve 225, and through second slot 232 of retractable sleeve 224, as shown in FIGS. 4A and 4B.

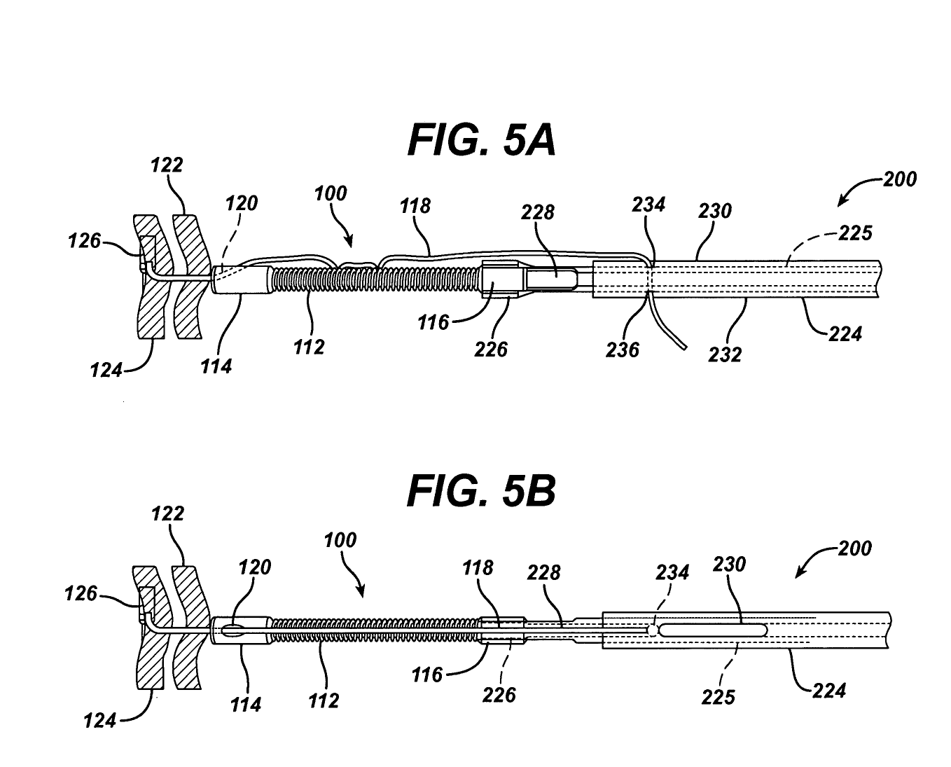

FIGS. 5A and 5B illustrate a side view and a top view, respectively, of the distal end of lock actuating device 200 with suture lock assembly 100 engaged therein in the cut state, which is described as follows.

Cut State:

In the cut state, the relative distance between the tip of actuating shaft 228 and the tip of retention jaw 226 is maintained, as set in the lock state. By using retract handle 214, which is attached to the proximal end of retractable sleeve 224, the physician retracts tip of retractable sleeve 224 in a direction away from the tip of retention jaw 226, which causes the position of hole 234 and slot 236 within retention sleeve 225 to change, relative to first slot 230 and second slot 232, respectively, such that suture 118 within hole 234 is cut as hole 234 passes underneath the edge of first slot 230, which has a ground edge suitable for cutting suture 118.

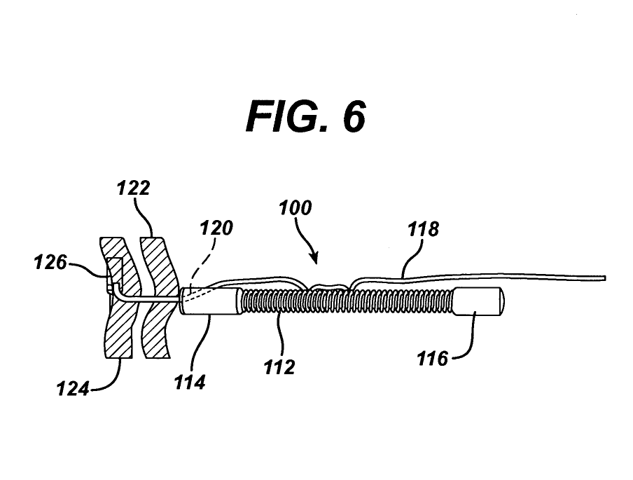

FIG. 6 illustrates a side view of suture lock assembly 100 in the release state, which is described as follows.

Release State:

In the release state, the physician manipulates the grasp of retention jaw 226 and proximal endcap 116 is released, which allows all instrumentation, such as lock actuating device 200 and the endoscope, as well as the surplus length of suture 118, to be removed. Extension spring 112 remains relaxed and, thus, the locking action upon suture 118 is maintained indefinitely within the patient.

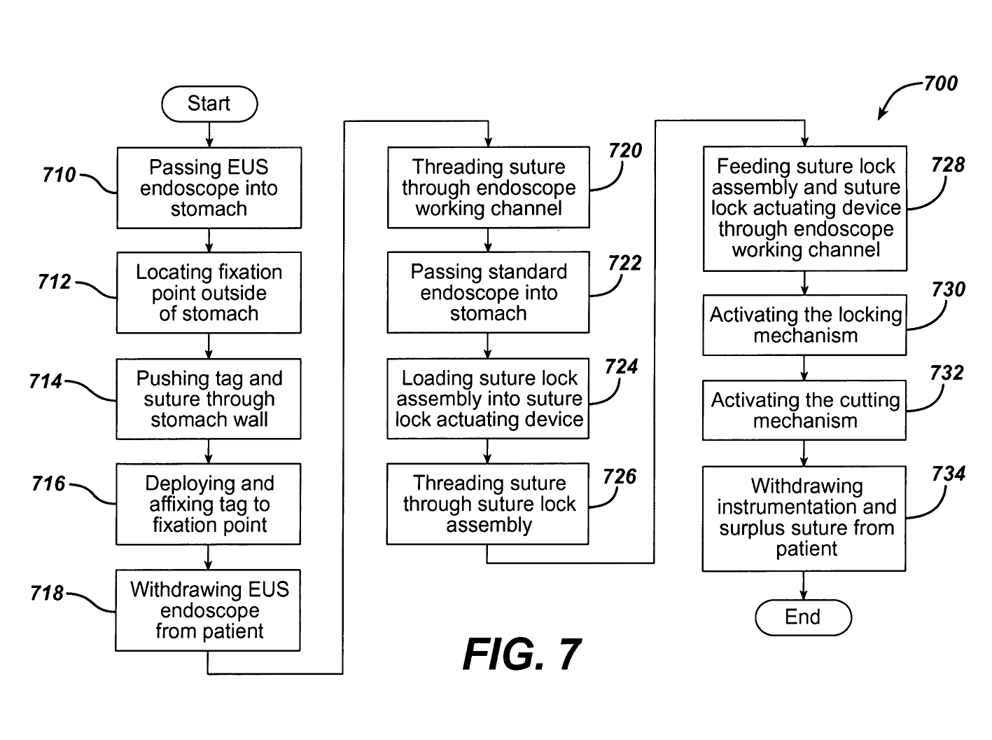

FIG. 7 illustrates a flow diagram of an example method 700 of using suture lock assembly 100 in combination with lock actuating device 200 in accordance with the invention. More specifically, method 700 provides an example of a posterior gastropexy procedure that uses suture lock assembly 100 of the present invention. The use of suture lock assembly 100 is not limited to a posterior gastropexy procedure; suture lock assembly 100 may be used in any of various, similar medical procedures. Furthermore, method 700 is not limited to a single suture 118 installed within suture lock assembly 100; a plurality of sutures 118 may be engaged within a single suture lock assembly 100.

At step 710, a physician passes an EUS endoscope through a patient's mouth and esophagus and into the stomach. Example EUS endoscopes include endoscope model GF-UC160P-AT8 manufactured by Olympus Europe (Hamburg, Germany) and endoscope model EG-3630U manufactured by Pentax Medical Company (Orangeburg, N.Y.). The working channel of the EUS endoscope is preloaded with a standard EUS needle, such as is manufactured by Wilson-Cook (Winston-Salem, N.C.), that serves as a carrier for a tag and suture, such as T-tag 126 and suture 118. Suture 118 may run either through the needle or outside the needle, but still inside the working channel of the EUS endoscope.

At step 712, under the guidance of the EUS endoscope, the physician locates and identifies structures outside the stomach wall and selects a fixation point, such as the median arcuate ligament.

At step 714, under the guidance of the EUS endoscope, the physician pushes the EUS needle, which is carrying T-tag 126 and suture 118, through the stomach wall, which is represented by first tissue 122 in FIGS. 3A, 3B, 4A, 4B, 5A, 5B, and 6.

At step 716, under the guidance of the EUS endoscope, the physician deploys and affixes T-tag 126, with suture 118 attached thereto, to the fixation point, such as to the median arcuate ligament, which is represented by second tissue 124 in FIGS. 3A, 3B, 4A, 4B, 5A, 5B, and 6.

At step 718, the physician withdraws the EUS endoscope and associated instrumentation from the patient, but leaves a length of suture 118 still threaded through the patient's gastroesophageal tract and anchored to second tissue 124 (e.g., median arcuate ligament). The length of suture 118 extends out of the patient's mouth and is accessible to the physician.

At step 720, the physician threads the length of suture 118 that is extending out of the patient's mouth into the distal end and out of the proximal end of the working channel of a standard endoscope that has a standard vision system (i.e., not an EUS endoscope).

At step 722, while holding tension on suture 118, the physician passes the endoscope through the patient's mouth and esophagus and into the stomach. A length of suture 118 is left extending out of the proximal end of the working channel of the endoscope and is accessible to the physician.

At step 724, the physician loads suture lock assembly 100 into the distal end of lock actuating device 200 and sets suture lock assembly 100 into the default state, as described in reference to FIGS. 3A and 3B.

At step 726, with suture lock assembly 100 in the default state and loaded into lock actuating device 200, the physician first threads the length of suture 118 that is extending out of the proximal end of the endoscope through hole 120 in distal endcap 114, then within the extended coils of extension spring 112 is wrapped multiple times around actuating shaft 228, then threaded through hole 234 of retention sleeve 225, then threaded through first slot 230 of retractable sleeve 224, then threaded through second slot 236 of retention sleeve 225 and, finally, threaded through second slot 232 of retractable sleeve 224, as shown in FIGS. 3A and 3B.

At step 728, while holding tension on suture 118, which is extending out of second slot 232 of retractable sleeve 224, the physician passes the suture lock assembly 100 and retractable sleeve 224 of lock actuating device 200 through the working channel of the endoscope and into the patient's stomach. Suture lock assembly 100 is sliding freely along suture 118 in the default state, until distal endcap 114 is firmly abutted against the inside of the stomach wall, which is represented by first tissue 122 in FIGS. 3A, 3B, 4A, 4B, 5A, 5B, and 6.

At step 730, having determined that the desired geometry change between the stomach and the median arcuate ligament (represented by first tissue 122 and second tissue 124) is achieved and while continuing to hold tension on suture 118, the physician sets suture lock assembly 100 into the lock state by using retention handle 218, as described in reference to FIGS. 4A and 4B, which causes the coils of extension spring 112 to relax and create a torturous path and, thus, clamp against suture 118, as shown in FIGS. 4A and 4B.

At step 732, having secured suture lock assembly 100 against first tissue 122 with suture 118, the physician sets suture lock assembly 100 into the cut state by using retract handle 214, as described in reference to FIGS. 5A and 5B, which causes suture 118 to be cut as hole 234 passes underneath the edge of first slot 230, which has a geometry suitable for cutting suture 118, as shown in FIGS. 5A and 5B.

At step 734, having secured suture lock assembly 100 against first tissue 122 and having cut suture 118, the physician releases retention jaw 226 from proximal endcap 116 of suture lock assembly 100, which allows all instrumentation, such as lock actuating device 200 and the endoscope, and the surplus length of suture 118, to be withdrawn from the patient, while suture 118 remains firmly clamped, as shown in FIG. 6. Method 700 ends.

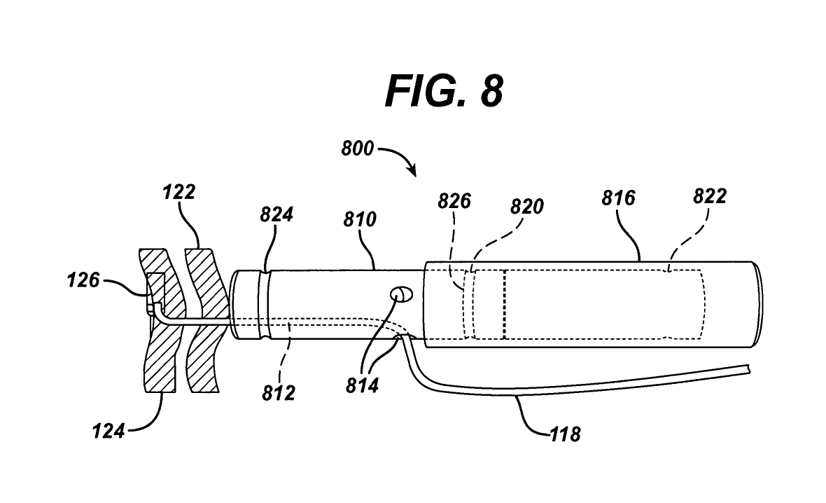

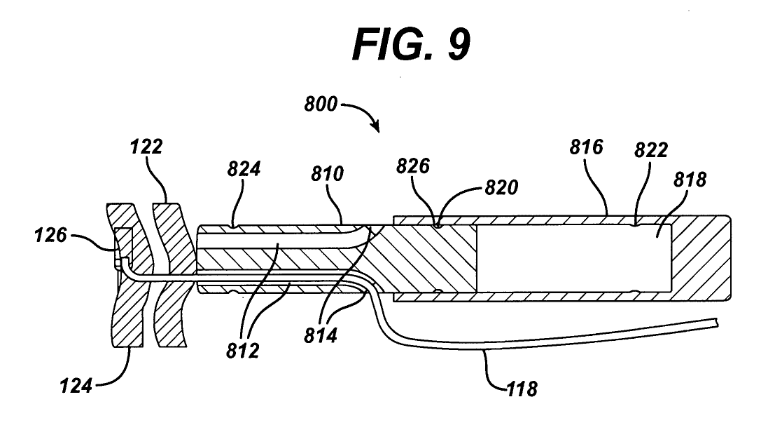

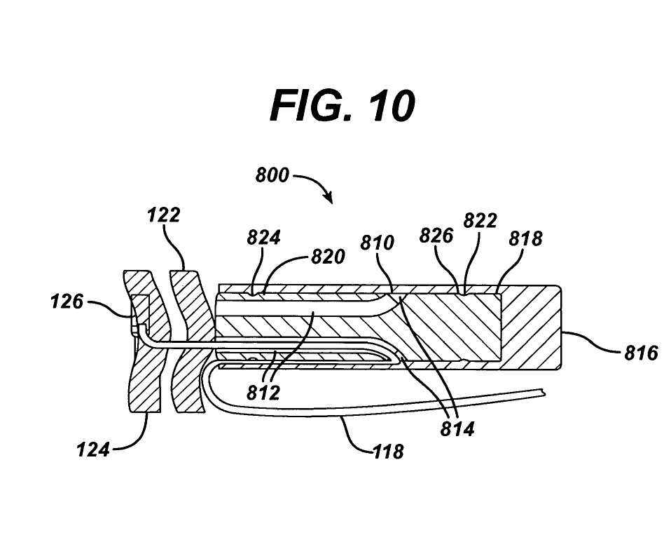

FIG. 8 illustrates a perspective view of a suture lock assembly 800 in accordance with a second embodiment of the invention. Suture lock assembly 800 includes a cylindrical-shaped lock body 810 that further includes a plurality of suture channels 812 that run therethrough, and which have an associated plurality of locking holes 814 arranged on the outer surface of lock body 810. Suture lock assembly 800 further includes a lock sleeve 816 that further includes a cavity 818 (shown in FIGS. 9 and 10) within which lock body 810 is inserted. Preferably there is a clearance of 0.001 inch or less between the outer surface of the lock body 810 and the inner surface of the lock sleeve 816. Lock body 810 further includes a first groove 824 and a second groove 826, which are detents formed around the outer perimeter of lock body 810. Lock sleeve 816 further includes a first locking ring 820 and a second locking ring 822, which are raised regions protruding from the inside perimeter of cavity 818 that are sized to lock within the detents formed by first groove 824 and second groove 826 of lock body 810.

Also shown in FIG. 8 is suture 118, which is anchored to second tissue 124 with T-tag 126 passes through first tissue 122 and into one of the suture channels 812, and exits lock body 810 via one of associated locking holes 814. Only a small portion of the distal end of lock body 810 is inserted into cavity 818, such that locking holes 814 are not within cavity 818 of lock sleeve 816. Lock body 810 and lock sleeve 816 are formed of, for example, molded plastic or stainless steel.

FIG. 9 illustrates a cross-sectional view of a suture lock assembly 800 and shows suture 118 passing through one of the suture channels 812 and existing lock body 810. FIGS. 8 and 9 are representative of suture lock assembly 800 in the default, unlocked state wherein one or more sutures 118 may be threaded freely through lock body 810. In the default or unlocked state first locking ring 820 of lock sleeve 816 is engaged within second groove 826 of lock body 810, as shown in FIGS. 8 and 9.

FIG. 10 illustrates a cross-sectional view of a suture lock assembly 800 in a locked state wherein or more sutures 118 is threaded through lock body 810 and locked therein. More specifically, in the lock state, lock sleeve 816 is pushed over the entire length of lock body 810, such that suture 118 is clamped between the outer surface of lock body 810 and the wall of cavity 818 of lock sleeve 816, after which any surplus suture 118 material is cut, which leaves suture lock assembly 800 secured against first tissue 122. In order to achieve the locked state enough force is applied to lock sleeve 816 against lock body 810 such that first locking ring 820 of lock sleeve 816 disengages from within second groove 826 of lock body 810. In doing so, lock sleeve 816 slides upon lock body 810 until first locking ring 820 and second locking ring 822 are engaged within first groove 824 and second groove 826, respectively, of lock body 810, as shown in FIG. 10. The mechanical features of suture lock assembly 800 for coupling lock sleeve 816 to lock body 810 are exemplary only and are not limited to first locking ring 820, second locking ring 822, first groove 824, and second groove 826. Any well-known coupling method that allows a default and lock state by sliding lock sleeve 816 upon lock body 810 may be used.

The overall diameter of suture lock assembly 800 is suitably small enough to allow it to pass through the working channel of various endoscopic and ultrasound devices, which is typically between 2.8 and 3.4 mm in diameter. See Table 2 for example dimensions of suture lock assembly 800.

TABLE 2
Example dimensions of suture lock assembly 800

| | Example Dimension |
|---|---|
| Lock body 810 length | 0.35 in |
| Lock body 810 outside diameter | 0.07 in |
| Suture channels 812 diameter | 0.015 in |
| Lock sleeve 816 length | 0.38 in |
| Lock sleeve 816 inside diameter | 0.07 in |
| Suture lock assembly 800 overall | 0.39 in |
| length when locked | |

The method of using suture lock assembly 800, in combination with suture 118, T-tag 126, first tissue 122, and second tissue 124, is generally the same as described in FIG. 7, in reference to suture lock assembly 100, in that it is fed down the working channel of an endoscope and into, for example, a patient's stomach, in much the same manner. However, suture lock assembly 800 requires no special actuating device; instead, it may be pushed through the working channel of an endoscope with, for example, the tip of a standard catheter. Additionally, its use differs from suture lock assembly 100, in that suture lock assembly 800 requires separate instrumentation for cutting the one or more sutures 118 engaged therein.

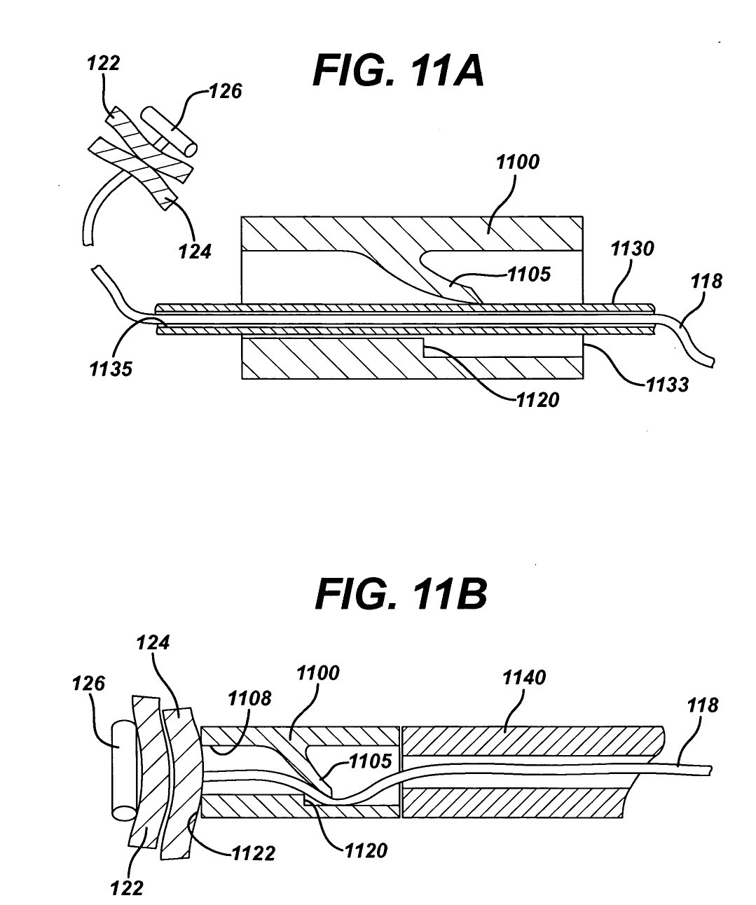

FIGS. 11A and 11B illustrate an alternative locking device similar in function to those previously mentioned. The locking device of FIGS. 11A and 11B are designed to lock onto suture 118, when used in conjunction with the endoscope. The locking device of FIGS. 11A and 11B may be placed on suture 118 attached to T-tag 126, that has been placed through first tissue 122 and second tissue 124 using the previously-described technique.

This embodiment comprises a tubular sleeve 1100, a flap 1105, and a detent 1120. Tubular sleeve 1100 may have an outer diameter of about 2.6 mm and an inner diameter of about 1 mm, and may be injection molded from a suitable polymer, such as polycarbonate, as a single piece or as separate pieces which are then fused together to form a unitary structure. In a resting state, flap 1105 is biased toward contact with detent 1120. Therefore, to load suture 118 into tubular segment 1100, an introducer 1130 may be used to create space between flap 1105 and detent 1120 as shown in FIG. 11A. Introducer 1130 may be placed into tubular segment 1100 by pushing from a distal end 1122 of tubular segment 1100, so that flap is moved away from detent 1120. Suture may be placed through a central lumen 1135 of introducer 1130, so that ultimately suture 118 is positioned within tubular segment 1100. Introducer 1130 is then removed by pulling it out of tubular segment 1100 from a proximal end 1133, so that introducer 1130 is not trapped between tubular segment 1100 and second tissue 124.

After introducer 1130 is removed, tubular segment 110 may be pushed along suture 118 toward second tissue 124 with a pusher 1140 especially designed for that purpose, as shown in FIG. 11B. Tension on suture 118 acts to pull flap 1105 partially away from detent 1120 during advancement. When distal end 1122 of tubular segment 1100 reaches second tissue 124, pusher 1140 is withdrawn and flap 1105 traps suture 118 against detent 1120 so that tubular segment 1100 is held securely in place.

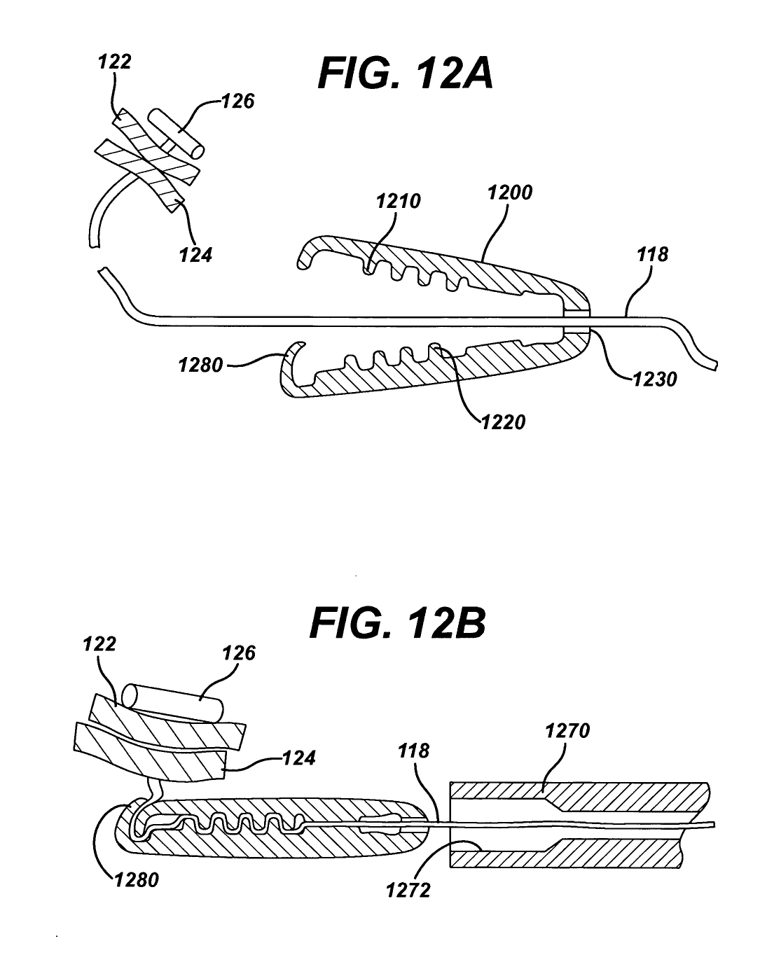

FIGS. 12A and 12B show another alternative concept for locking onto suture 118. FIG. 12A shows a perspective view of a clip 1200 comprising a first gripping surface 1210, a second gripping surface 1220, an opening 1230, and a clasp 1240. In a default state, clip 1200 is open as shown in FIG. 12A, so that suture 118 can pass freely through opening 1230. Clip 1200 may be placed on suture 118 attached to T-tag 126, that has been placed through first tissue 122 and second tissue 124 using the previously-described technique. Clip 1200 may be placed onto suture 118 so that clasp 1280 is directed toward second tissue 124 and opening 1230 is directed toward the user. Clip 1200 may be pushed down suture 118 using a long flexible tube, such as an endoscope.

To lock clip 1200 onto suture 118, a horn 1270 including a tapered surface 1272 may be used to apply force at a proximal end of clip 1200, so that first gripping surface 1210 mates with second gripping surface 1220 to securely hold onto suture 118, while clasp 1280 holds clip 1200 closed. Clip 1200 may be made from any suitable polymer material, such as nylon. Clip 1200 may be injection molded as a unitary piece with a “living hinge” that biases the part to an open position in which a first gripping surface 1210 is held away from second gripping surface 1220 in a default open state or assembled from multiple pieces.

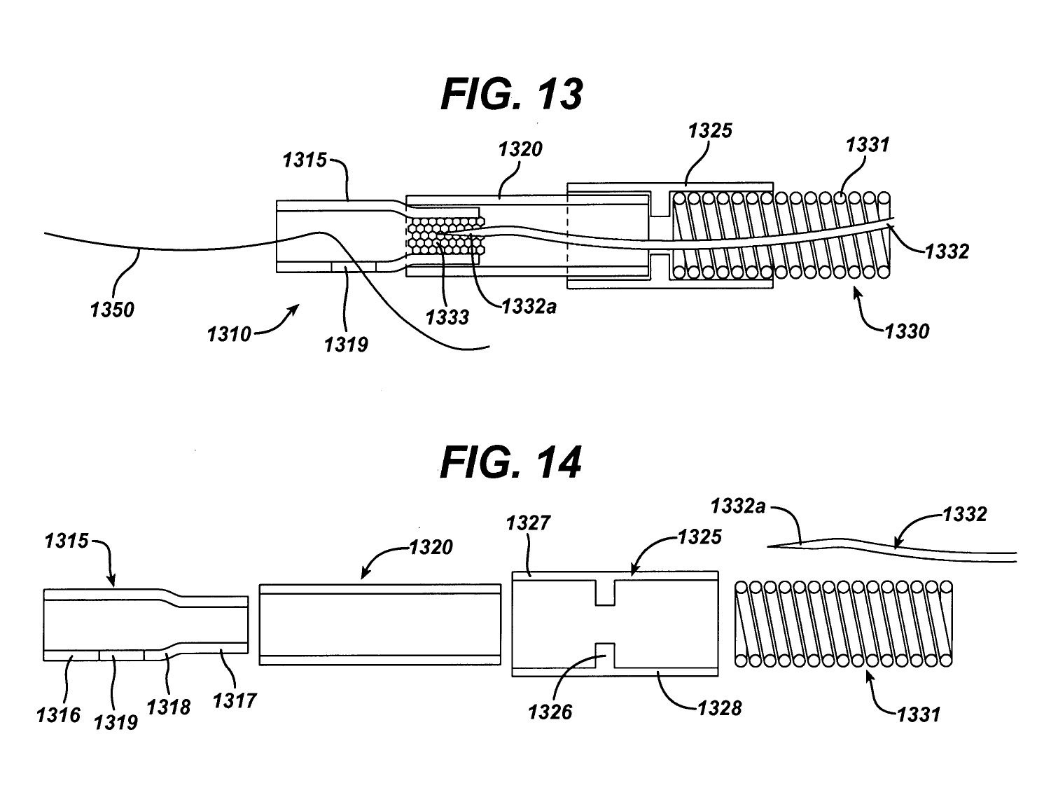

A description will now be given of the locking device shown in FIGS. 13, 14, 15 and 16a through 16g. FIGS. 13 and 14 show the head 1310 of a locking device comprising, successively along its length, an inner locking tube 1315, preferably of metal, an outer locking tube 1320, preferably of a plastics material, and a connecting tube 1325, preferably of metal, which connects the tube 1320 to the wire-wound sheath 1331 of a Bowden cable 1330. The inner wire of the Bowden cable is shown as 1332. FIG. 13 also shows part of a suture 1350 which the device is to lock.

Considering now the individual components in more detail, the tube 1315 can conveniently be formed of the same type of tubing as that used for hypodermic needles, but formed to have a wider distal portion 1316 and a narrower proximal portion, smoothly connected by a tapering portion 1318. The taper is sufficiently steep to prevent someone handling the device accidentally pushing the tube into the tube 1320. An aperture 1319 is formed in the wall of the larger diameter portion 1316. If desired, a pair of such apertures may be provided, for example offset from one another about the axis of the tube by 180°. This makes it easier to use the device to lock together a plurality of sutures, or a lock a single suture to itself at a plurality of points, possibilities which are mentioned again below. The suture 1350 is shown in FIG. 13 passing through the aperture 1319. The end edges of the tube 1315 are preferably rounded not shown to reduce the risk of the patient or, indeed, the suture 1350 being cut inadvertently.

The tube 1320 is formed of a material which is able to deform to the requisite extent during operation of the device, as will be explained below. It is therefore preferably formed of a plastics material. One material which may be used is polyethylene, though other plastics materials such as polyethyl ethyl ketones may be preferable, as they have less tendency to creep over time as a result of the warmth of the patient's body. The tube 1320 is a simple cylinder, the internal diameter of which is such that the smaller diameter portion 1317 of the tube 1315 can be held therein by an interference fit. For example, the external diameters of the portions 1316 and 1317 may be 1.7 mm and 1.47 mm respectively, and the internal diameter of the tube 1320 may be 1.4 mm.

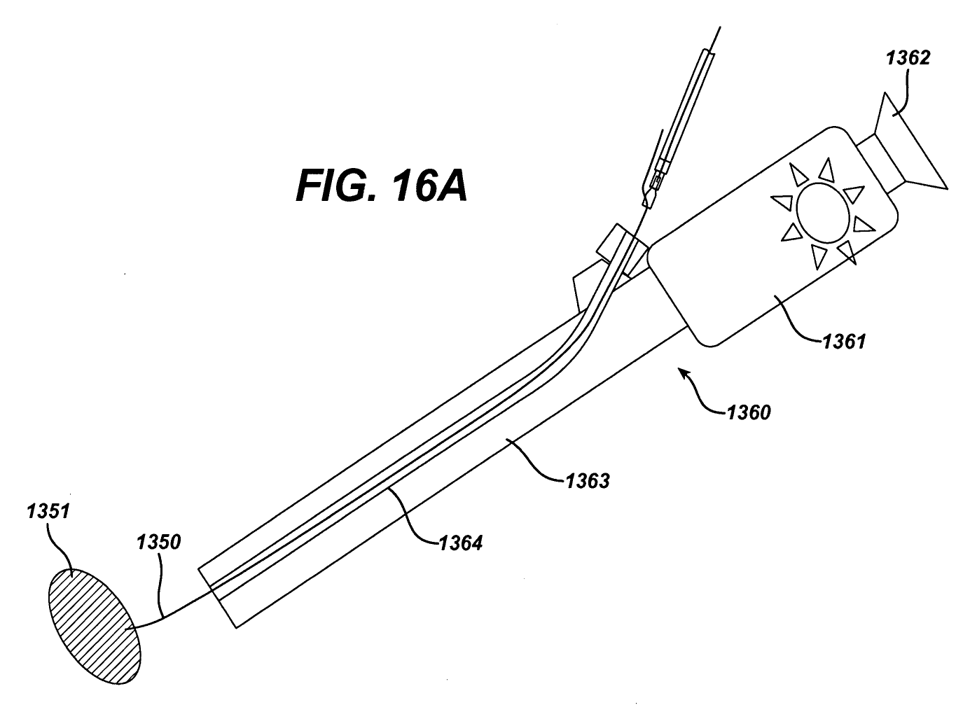

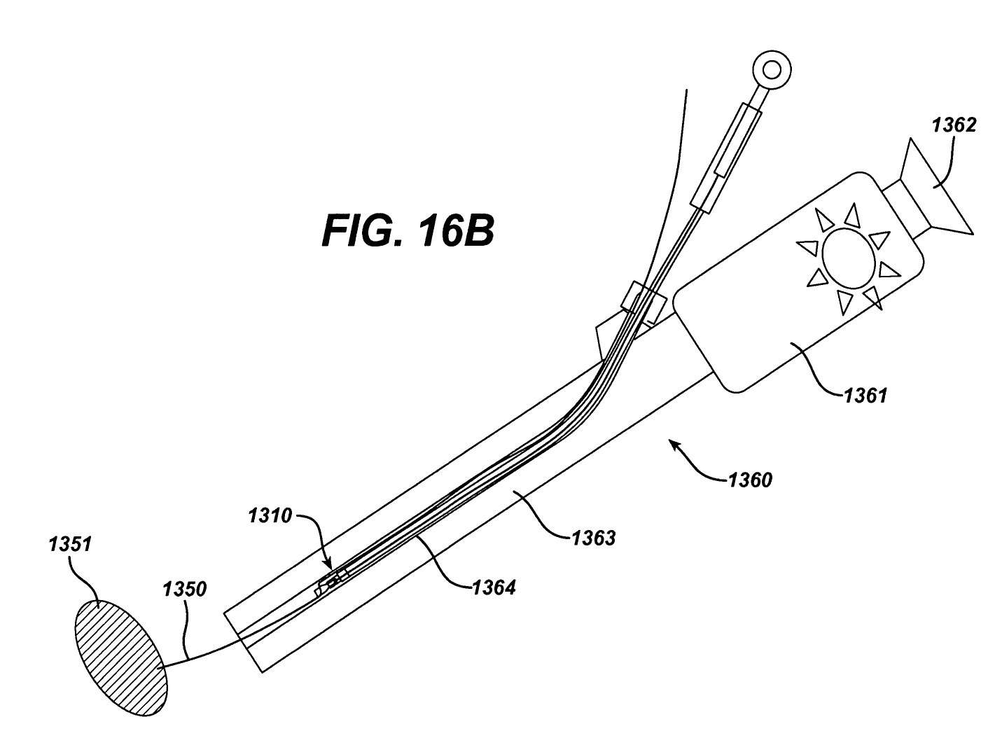

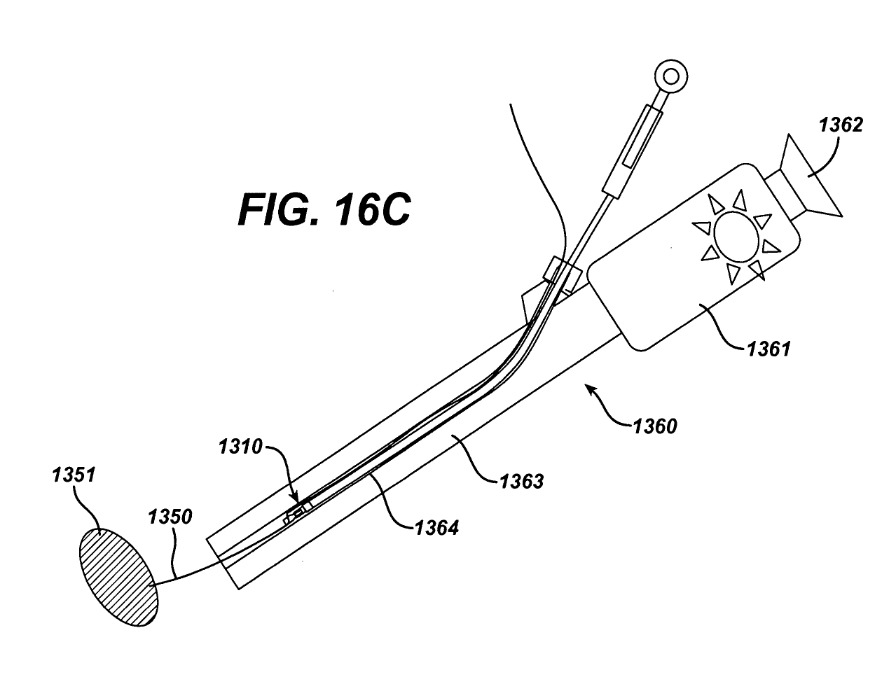

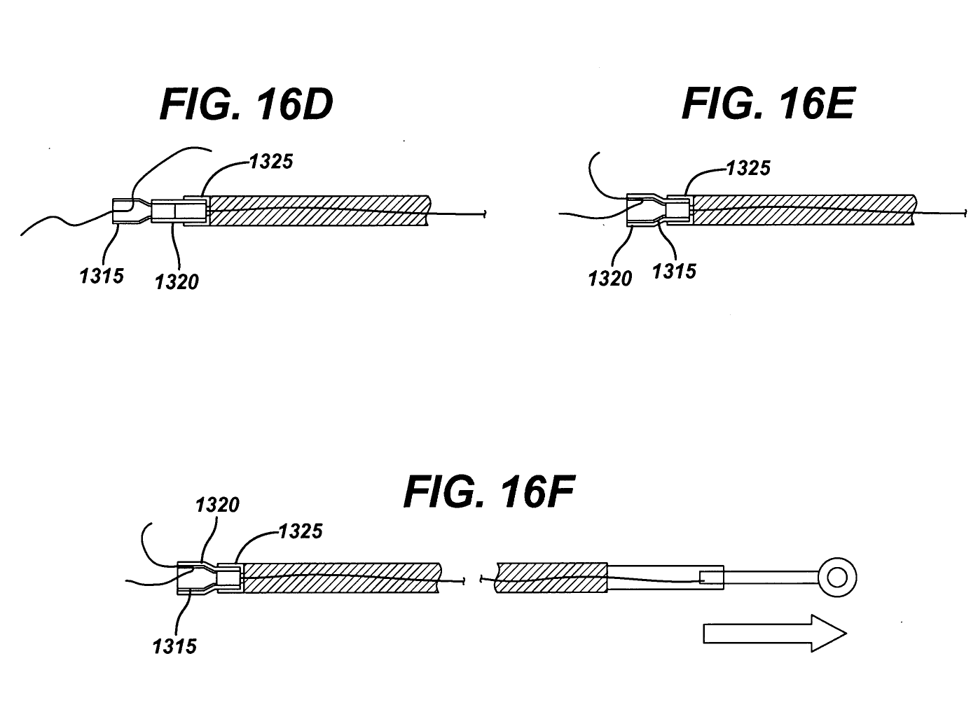

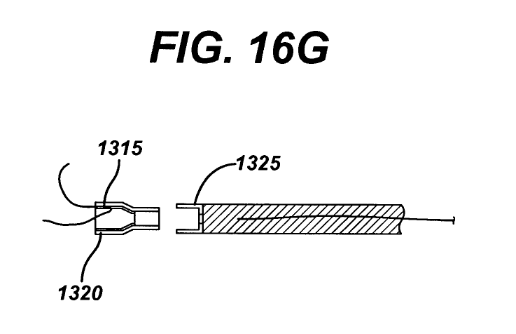

The connecting tube 1325 is preferably formed of metal, for example of stainless steel. It is in the form of a cylinder with an internally projecting boss 1326 provided, preferably integrally, with the remainder of the cylinder, part way along its length. The internal diameter of the distal portion 1327 of the tube 1325 is such that the end portion of the plastic tube 1320 can be received therein. However, it should not be too tight a fit therein, since at the end of the locking procedure see below the tubes 1320 and 1325 have to be separated from one another. The internal diameter of the proximal portion 1328 of the tube 1325, which may or may not be the same as the internal diameter of the distal portion 1327, is such that the wire-wound sleeve 1331 of the Bowden cable 1330 is an interference fit therein. It should be understood, however, that the sleeve 1331 could be additionally or alternatively connected by some other means to the connecting tube 1325, for example by an adhesive such as a cyanoacrylate adhesive. As will be apparent from what is said below, the connecting tube 1325 is intended to remain permanently fixed to the cable 1330, without there being need for any movement therebetween, so the connection between them can, and should, be made by as secure a means as possible. It should also be mentioned that in FIGS. 16a to 16g, showing the steps in the operation of the device, the connecting tube 1325 has been shown with the portion 1328 omitted and the end of the cable 1330 butt-joined to the boss 1326. This has been done purely for ease of illustration in FIGS. 16e to 16g, and it is to be understood that, in practice, the connecting tube 1325 and cable 1331 would normally be connected as shown in FIG. 13, or at least by some other means more secure than a butt joint.

As mentioned above, the Bowden cable 1330 has a wire-wound sheath 1331 and an inner wire 1332. The wire 1332 has a tapered distal end portion 1332a which is releasably connected to the tube 1315. In the illustrated embodiment the wire portion 1332a is connected to the smaller diameter portion 1317 of the tube 1315 by solder 1333, represented purely diagrammatically by the illustrated hexagons. In this way a frangible connection is formed between the wire portion 1332a and the tube 1315. Alternatively, however, some other form of releasable connection could be used. For example, the wire 1332 and tube 1315 could carry respective components of a ball and detent system which, in a similar way to the solder, will provide a connection between the wire and the tube which holds until a certain level of tension is applied, but which permits separation between them once that level of tension is exceeded.

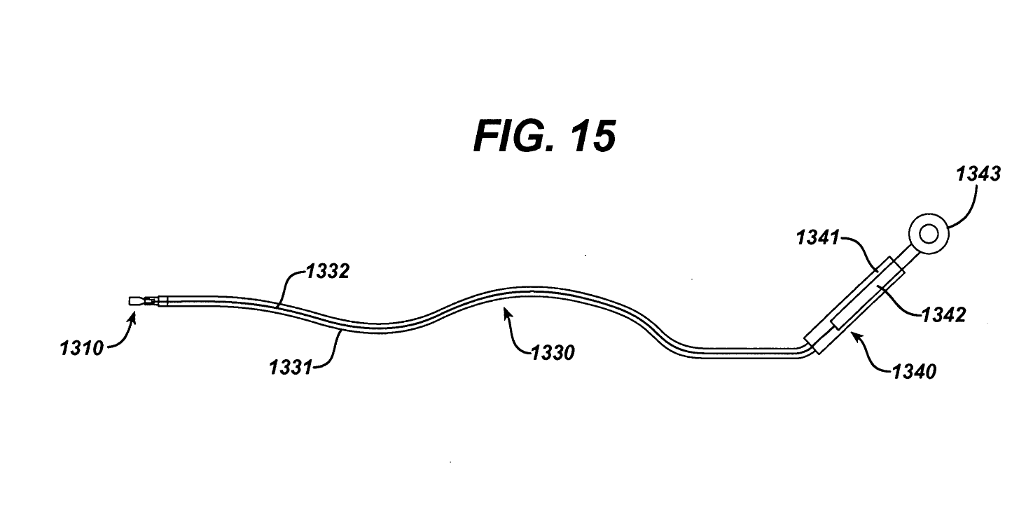

FIG. 15 shows, on a much smaller scale than FIGS. 13 and 14, the head 1310 of the locking device mounted on one end of the Bowden cable 1330, and a handle 1340 connected to the other end of the Bowden cable. The handle 1340 comprises an outer member 1341 to which the sheath 1331 of the Bowden cable is attached, and an inner member 1342 which is slideable within the outer member 1341, and to which the wire 1332 of the Bowden cable is attached. The inner member 1342 can be moved with respect to the outer member 1341 by means of a portion 1343 which can be grasped by a user. It will be understood the handle 1340 is shown only diagrammatically, and that the person skilled in the art could readily substitute for what is shown a handle of a more ergonomic design.

The operation of the locking device described above will now be described with reference to FIGS. 16a to 16g, which show successive steps in the operation.

The starting point for the locking procedure, as shown in FIG. 16a, is that one end of the piece of elongate suture material 1350 has become anchored in an area of tissue 1351 within the body of a patient, either human or animal, for example by a sewing operation. The manner in which this sewing is effected does not form part of the present invention, and there are various known methods by which such sewing can be effected.

However, although it is not essential, it is preferred (and the device of the present invention is designed so that this is possible) that in the subsequent locking operation the suture 1350 should pass from the tissue, up through the biopsy channel of the endoscope, and thence to the exterior of the patient. This will automatically be the case if the suture was already present in the biopsy channel during the sewing procedure. If this was not the case then the endoscope tube can to be threaded onto the suture 1350 and passed down into the patient, so that its distal end is adjacent the tissue 1351, before the locking operation begins. In either event, with the suture 1350 passing through the biopsy channel, the end of the suture that is outside the patient is threaded through the locking device, passing into the interior of the tube 1315 at its distal end, and out of that tube through the aperture 1319, or one of the apertures 1319, as the case may be.

The arrangement is now as shown in FIG. 16a. It should be noted that FIG. 16a shows a conventional flexible endoscope 1360, which comprises a head 1361 having a viewing opening 1362 through which an image produced by the endoscope can be viewed by the user, and an elongate flexible tube 1363 which includes the biopsy channel 1364. Typically this channel has a diameter of between 2.8 mm and 3.4 mm, and constitutes the working channel of the endoscope. As will be apparent from what is said below, the Bowden cable and locking device must be of small enough diameter to pass into and through the biopsy channel.

With the arrangement as in FIG. 16a, the proximal end of the suture 1350 is then pulled, so as to take up the slack, and the locking device is slid down over it in a direction towards the biopsy channel. With a tension still being exerted on the suture, the locking device is slid further along it, so that it enters the channel 1364 of the endoscope 1360, whereafter pushing on the outer member of the handle attached to the sleeve of the Bowden cable causes the locking device and cable both to travel down the biopsy channel. The position is then as shown in FIG. 16b.

The locking device is then pushed further along the channel 1364, emerging at the distal end thereof, so that it is located adjacent the area of tissue 1351, as shown in FIG. 16c.

Once the locking device has emerged from the distal end of the biopsy channel, and is adjacent the tissue 1351, locking is caused to take place. This is done by the user pulling on the handle member 1343 so as to draw the tube 1315 rearwards with respect to the other elements of the locking device. This moves the tube 1315 from the position shown in FIG. 16d to the position shown in FIG. 16e. In this movement, the tube 1315 is forced into the tube 1320, deforming the latter as it does so and trapping the suture between the other wall of the portion 1316 of the tube 1315 and the adjacent portion of the inner wall of the tube 1320.

The locking device is so designed that the transition to the state shown in FIG. 16e can be achieved by applying a relatively low tension to the wire 1332 of the Bowden cable. The tension required is low enough that the connection between the wire portion 1332a and the tube 1315 remains intact. The next step is to apply a higher tension which, as shown in FIG. 16f, causes the wire portion 1332a to break away from its connection to the tube 1315. In the illustrated embodiment this involves the breaking of the connection provided by the solder 1333.

Once this connection has been broken, tension is applied to the sheath of the Bowden cable to cause the portion 1327 of the locking cylinder 1325 to slide off the tube 1320, as shown in FIG. 16g. Continued pulling on the Bowden cable removes it completely from the patient's body. Once this has been done, all that remains inside the body is what is shown at the left hand side of FIG. 16g, namely the combination of tubes 1315 and 1320, one inside the other, with the suture 1350 locked between them. It is to be noted that the circumferential wall of the tube 1315 is entirely surrounded by the plastics tube 1320. This makes it possible, by choosing a suitable material for the tube 1320, to minimise trauma to the neighbouring tissues.

Various modifications can be made to the illustrated embodiment, in addition to those already mentioned above. For example, rather than use a handle of the general form indicated by reference numeral 1340, the proximal end of the Bowden cable could be attached to a winding device, by means of which tension can be exerted on the wire 1332 thereof by winding it onto a spool. This makes it possible to retract the wire to an unlimited extent, which is not possible using a handle with inner and outer members telescopically arranged. Also, although the device is shown being used to lock a single suture, it could, without modification, be used to lock a plurality of sutures together, or to lock a single suture to itself at a plurality of points.

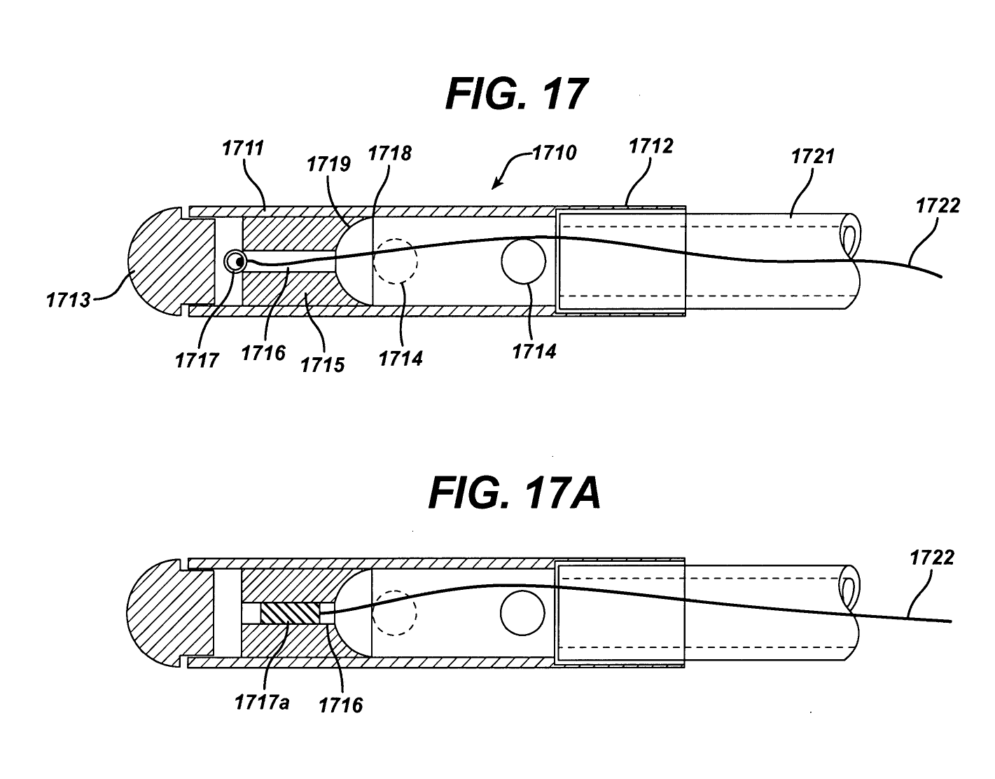

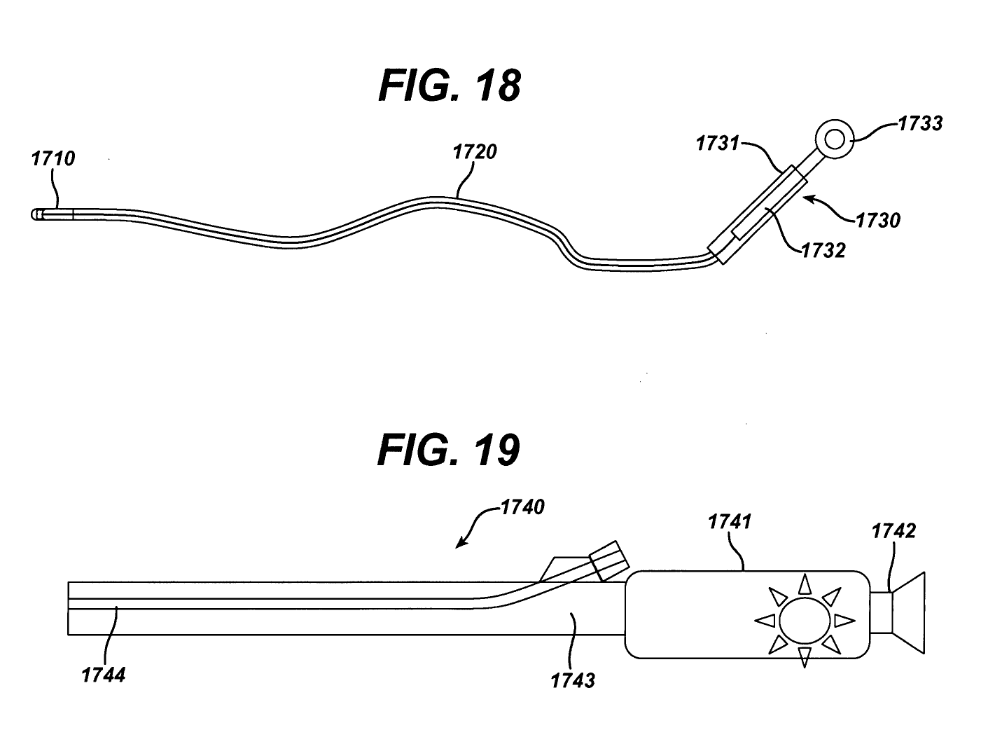

A description will now be given of the cutting device shown in FIGS. 17, 17a, 18, 19 and 20a through 20f. As can be seen from a consideration of FIGS. 17 and 18, the cutting device comprises a cutter head 1710 mounted on one end of a wire-bound cable (a Bowden cable) 1720. The other end of the cable 1720 is connected to a handle 1730.

The cutter head 1710 comprises a cylinder 1711 having a portion 1712 at its proximal end which is of larger internal diameter and to which the sheath 1721 of the Bowden cable 1720 is fixedly attached. At its distal end the cylinder 1711 is closed by a nosepiece 1713 having a rounded outer surface to make it easier to introduce the cutter head into and through the biopsy channel of an endoscope (as described further below). The nosepiece 1713 is shown as being an entity distinct from the cylinder 1711, and it is shown as having a hemispherical outer surface. However, the surface could have some other suitable shape, and it could be provided by an integral portion of the cylinder 1711 itself. Two apertures 1714 are formed through the wall of the cylinder, the apertures being spaced longitudinally from one another and offset from one another around the circumference of the cylinder. In the illustrated embodiment they are offset from one another by 180 degrees, as will be apparent from the ensuing description of the operation of the device. However, some other angle of circumferential offset could be used instead.

A cylindrical cutting member 1715 is slideably received within the cylinder 1711. The inner wire 1722 of the Bowden cable 1720 has its distal end attached to the cutting member 1715. One way of effecting this attachment is, as shown in the illustrated embodiment, to provide the cutting member 1715 with a longitudinal bore 1716, the wire 1720 being threaded through the bore and being provided with an enlarged portion 1717 at the distal end, of a size too great to allow it to be withdrawn through the bore. The proximal end of the cutting member 1715 has a cutting edge 1718 formed thereon, for example by forming a hemispherical recess 1719 within the proximal end portion of the cutting member 1715. Preferably, the wire 1722 is stiff enough to allow the cutting member to be pushed back and thereby reset, so that it can be used repeatedly. FIG. 17a shows a modified form of the attachment between the distal end of the wire 1722 and the cutting member 1715, which is more suitable when the wire is relatively stiff. This employs a block 1717a which is attached, for example by welding, gluing or as a force fit, within the bore 1716, the distal end of the wire 1722 being itself attached to the block 1717a by, for example, crimping.

As show in FIG. 18, the handle 1730 comprises an outer member 1731 to which the sheath 1721 of the Bowden cable is attached, and an inner member 1732 which is slideable within the outer member 1731, and to which the wire 1722 of the Bowden cable is attached. The inner member 1732 can be moved with respect to the outer member 1731 by means of a portion 1733 which can be grasped by a user. It will be understood that the handle 1730 is shown only diagrammatically, and that the person skilled in the art could readily substitute for what is shown a handle of a more ergonomic design.

FIG. 19 shows diagrammatically a flexible endoscope 1740 with which the cutting device of the present invention may be used. This comprises, as is conventional, a head 1741 having a viewing opening 1742 through which an image produced by the endoscope can be viewed by the user, and an elongate flexible tube 1743 which includes a biopsy channel 1744, otherwise more generally referred to as the working channel. Typically, this channel has a diameter of around 2.8 mm to 3.4 mm, and the Bowden cable 1720 and cutting device 1710 are of small enough diameter to pass into and through the biopsy channel, in the procedure for use which will now be explained.

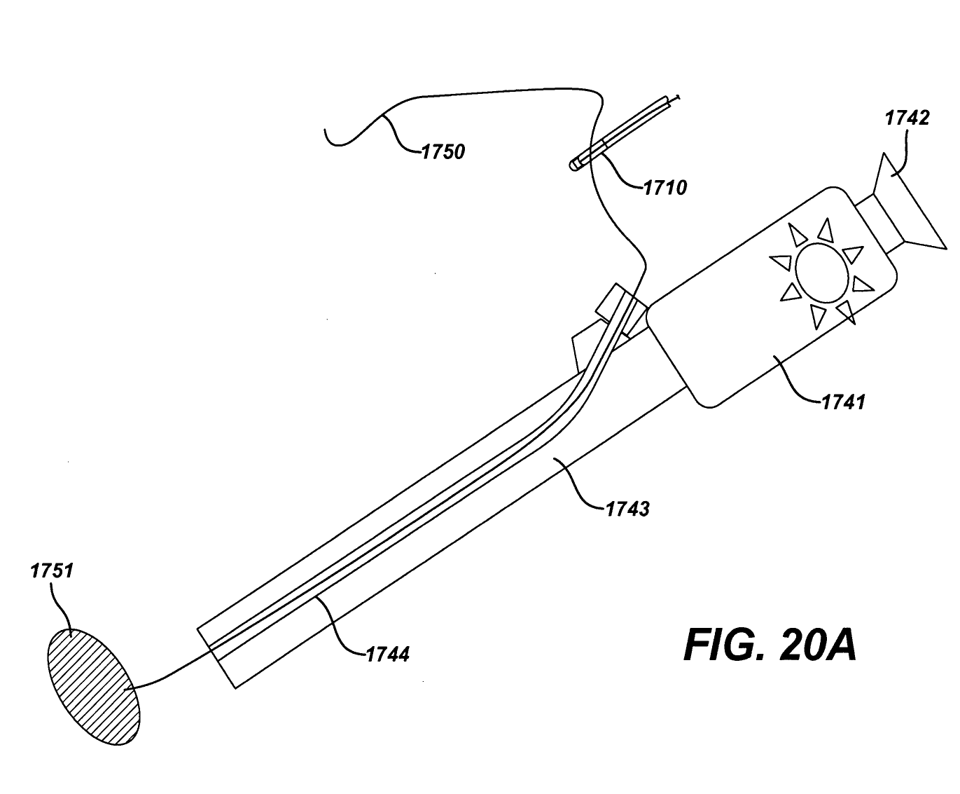

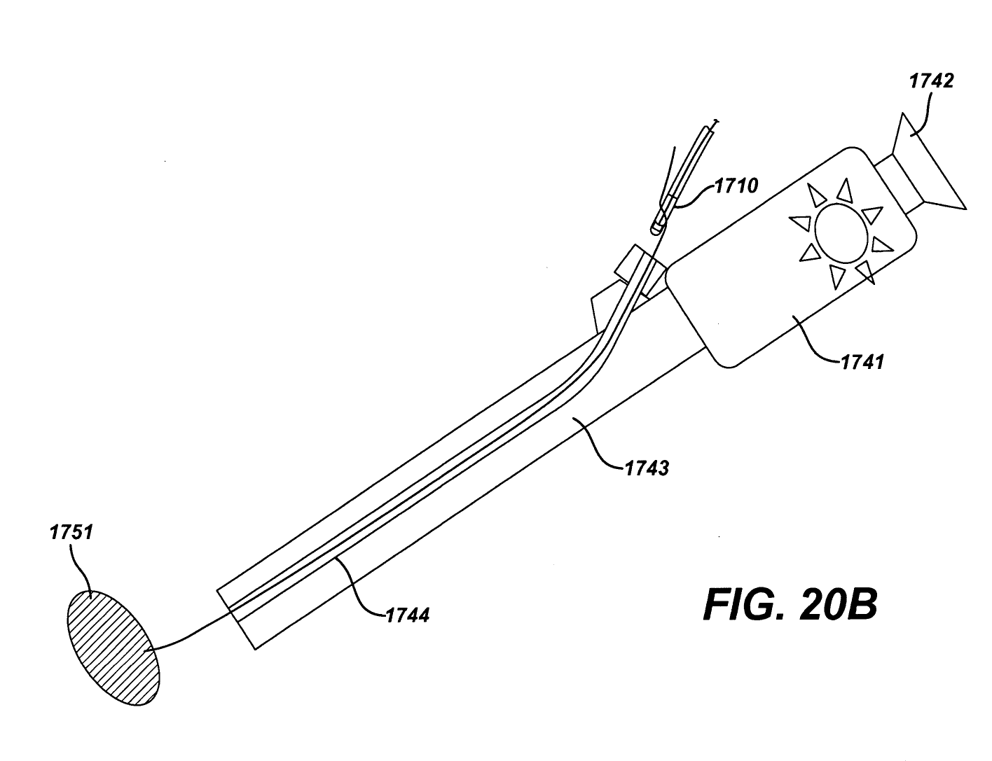

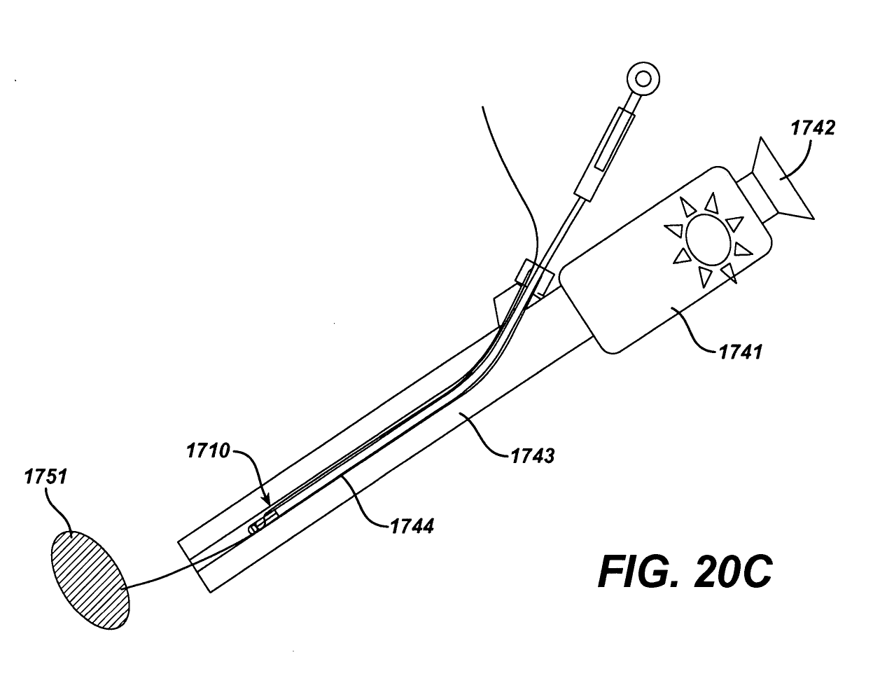

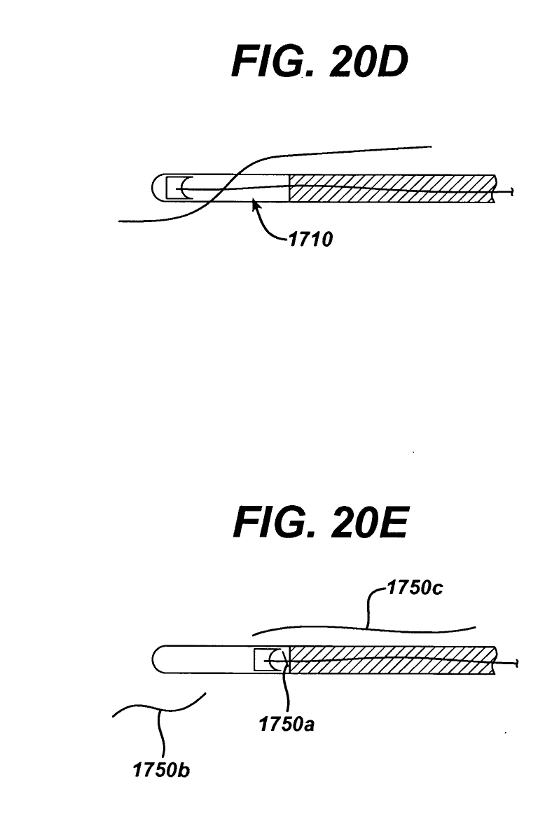

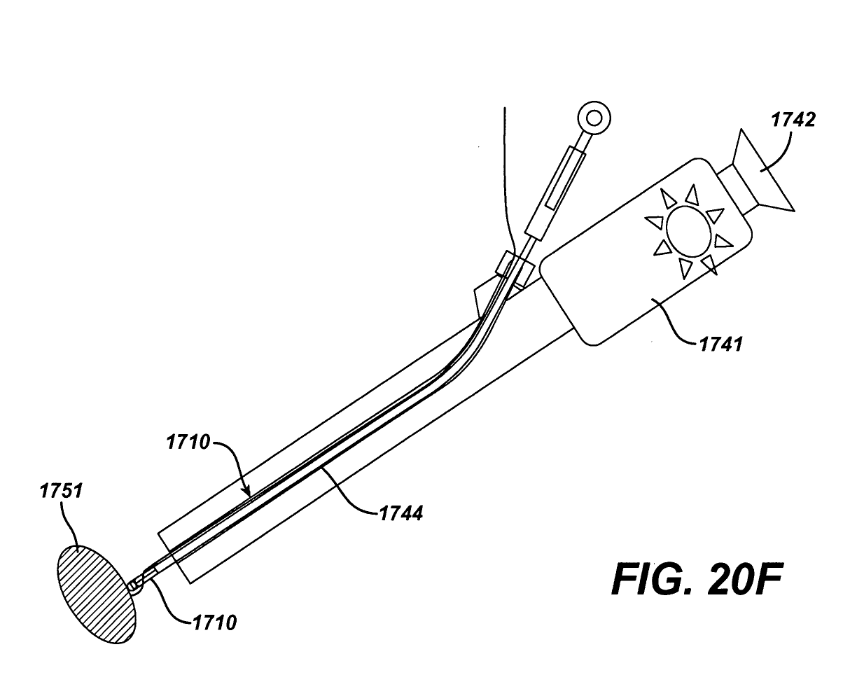

FIGS. 20a through 20f show successive steps in carrying out a cutting procedure using the device of the present invention. The starting point, as shown in FIG. 20a, is that one end of a piece of elongate suture material 1750 has become anchored in tissue 1751 within the body of a patient, either human or animal, for example by a sewing operation. The manner in which this sewing is effected does not form part of the present invention, and there are various known methods by which such sewing can be effected. However, it is a preferred (and the device of the present invention is designed so that this is possible) that in the subsequent cutting operation that the suture 1750 should pass from the tissue, up through the biopsy channel of the endoscope, and thence to the exterior of the patient. This will automatically be the case if the suture was already present in the biopsy channel during the sewing procedure. If this was not the case then the endoscope tube can be threaded onto the suture 1750 and passed down into the patient, so that its distal end is adjacent the tissue 1751 before the cutting operation begins. In either event, with the suture 1750 passing through the biopsy channel 1744, the end of the suture that is outside the patient is threaded through the cutting head 1710, passing into the interior of the cylinder 1711 through the more distal of the two apertures 1714, and out of the cutting head through the other of the apertures 1714. This can be done purely with the user's figures or with the aid of a conventional needle-threading device. The arrangement is now as shown in FIG. 20a.

The proximal end of the suture is then pulled, so as to take up the slack, and the cutting head is slid down over it in a direction towards the biopsy channel of the endoscope. The position is then as shown in FIG. 20b.

With a tension still being exerted on the suture, the cutting head is slid further along it, so that it enters the channel of the endoscope, whereafter pushing on the outer member of the handle attached to the sleeve of the Bowden cable causes the cutting head and cable both to travel down the biopsy channel. The position is then as shown in FIG. 20c.

Once the cutting head has emerged from the distal end of the biopsy channel, and is adjacent the tissue 1751, cutting is caused to take place. This is done by the user pulling on the handle member 1733 so as to draw the cutting element 1715 rearwards with respect to the cylinder 1711. The cutting surface 1718 of the cutting element 1715 thus slides across each of the apertures 1714 in turn, cutting the suture at the points where the suture passes respectively through those apertures. This leaves an off-cut 1750a within the cylinder 1711, a suture portion 1750b anchored to the tissue, and a relatively lengthy suture remnant 1750c running through the biopsy channel. Alternatively, the cutting element could be arranged to stop before it performs the second, more proximal, cut, in which case no off-cut 1750a would be produced. The suture need not be held under tension as the cutter element 1715 is passing the apertures 1714, provided the cutting element is in sufficiently close engagement with the adjacent cylinder wall to prevent the suture jamming therebetween instead of being cut. The position is now as shown in FIGS. 20e and 20f.

Finally, the cutting device and the remnant of suture 1750c are withdrawn from the biopsy channel of the endoscope.

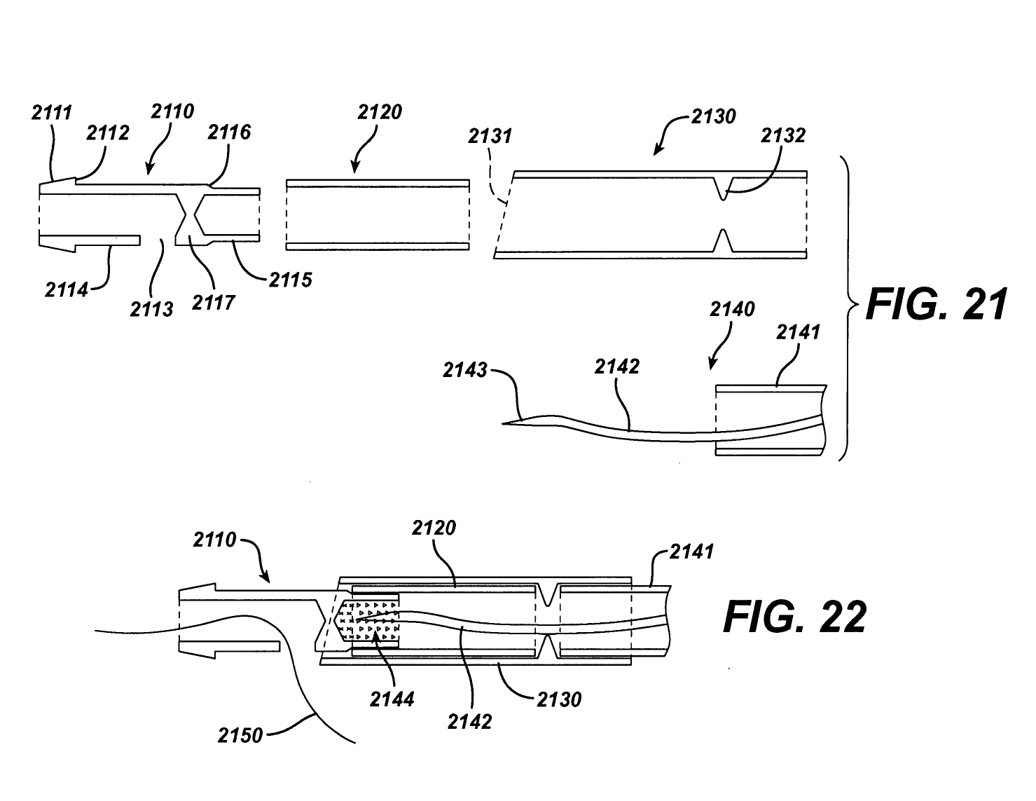

A description will now be given of the combined locking and cutting device shown in FIGS. 21, 22, 23, 24a, 24b and 25a through 25d. The device shown in FIGS. 21 and 22 comprises a piston 2110, preferably made of steel, a plastics inner tube 2120, an outer tube 2130, also made of steel, and a Bowden cable 2140. Each of these components, and their relationship to one another, will now be described.

The piston 2110 is generally cylindrical in shape. At its distal end it has an outwardly directed flange 2111 formed integrally therewith and tapering towards its distal end. The proximal end of the flange 2111 defines a cutting edge 2112. An aperture 2113 extends through the wall of the piston, the aperture being sized so that a suture which is to be locked and cut by the device can pass freely through it. The suture is shown in FIG. 22, and is denoted by reference numeral 2150. The piston can be regarded as having a distal portion 2114 with a first outer diameter, and a proximal portion 2115 with a second outer diameter which is less than the first diameter. The portions 2114 and 2115 are connected by an inclined portion 2116, which provides a transition between the first diameter and the second diameter. To provide additional stability, and to assist in the connection of the piston 2110 to the tube 2120 (for which see below), the interior of the piston may be spanned in the region of the junction of the portions 2114 and 2115 by a transverse wall 2117, the aperture 2113 being located distally of the wall 2117.

The inner tube 2120 has an internal diameter slightly smaller than the external diameter of the portion 2115 of the piston 2110, such that it can easily be force-fitted over the portion 2115. However, the outer diameter of the portion 2114 is larger than the internal diameter of the tube 2120 by a sufficient amount to avoid any likelihood of its being accidentally pushed over that portion. The tube 2120 is formed of a material which is able to deform to the requisite extent during operation of the device, as will be explained below. It is therefore preferably formed of a plastics material. One material which may be used is polyethylene, though other plastics materials such as polyethyl ethyl ketones may be preferable, as they have less tendency to creep over time as a result of the warmth of the patient's body.

The outer tube 2130 is preferably of steel, and is bevelled at its distal end 2131. It is connected to the inner tube by an interference fit. It should be understood, however, that the tube 2130 could be additionally or alternatively connected by some other means to the tube 2120, for example by an adhesive such as a cyanoacrylate adhesive. As will be apparent from what is said below, the outer tube 2130 is intended to remain permanently fixed to the tube 2120, without there being need for any movement therebetween, so the connection between them can, and should, be made by as secure a means as possible.

The tube 2130 has an inwardly directed flange 2132, which serves as an abutment for the proximal end of the inner tube 2120, and for the distal end of the wire-wound sheath 2141 of the Bowden cable 2140. The internal diameter of the proximal portion of the outer tube 2130, which may or may not be the same as the internal diameter of the distal portion thereof, is such that the wire-wound sleeve 2141 of the Bowden cable 2140 is an interference fit therein.

The Bowden cable 2140 further comprises an inner wire 2142, which terminates at its distal end in a tapered portion 2143. The portion 2143 is releasably connected to the piston 2110. In the illustrated embodiment the wire portion 2143 is connected to the smaller diameter portion 2115 of the tube 2110 by solder 2144. In this way a frangible connection is formed between the wire portion 2143 and the tube 2110. Alternatively, however, some other form of releasable connection could be used. For example, the wire 2142 and piston 2110 could carry respective components of a ball and detent system which, in a similar way to the solder, will provide a connection between the wire and the tube which holds until a certain level of tension is applied, but which permits separation between them once that level of tension is exceeded.

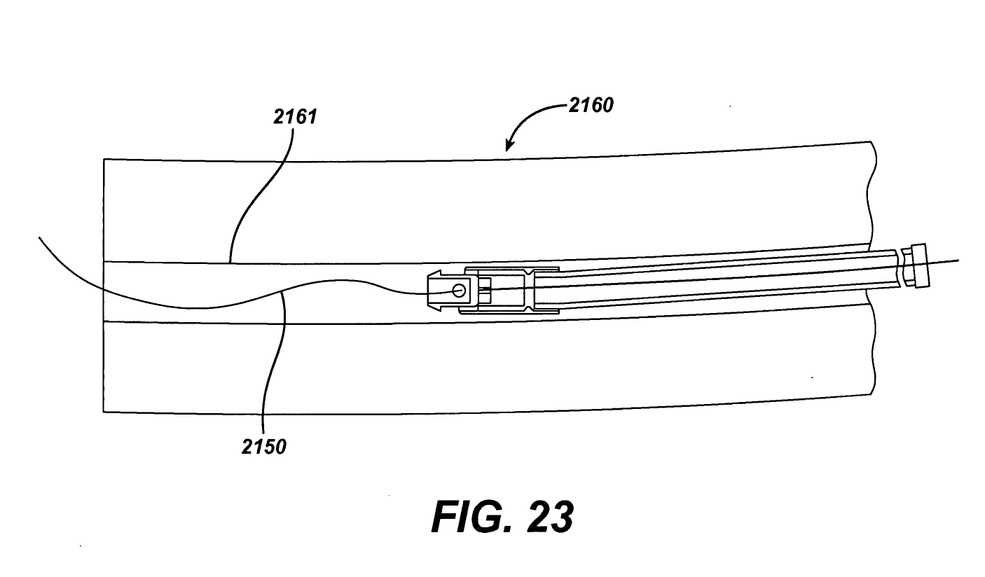

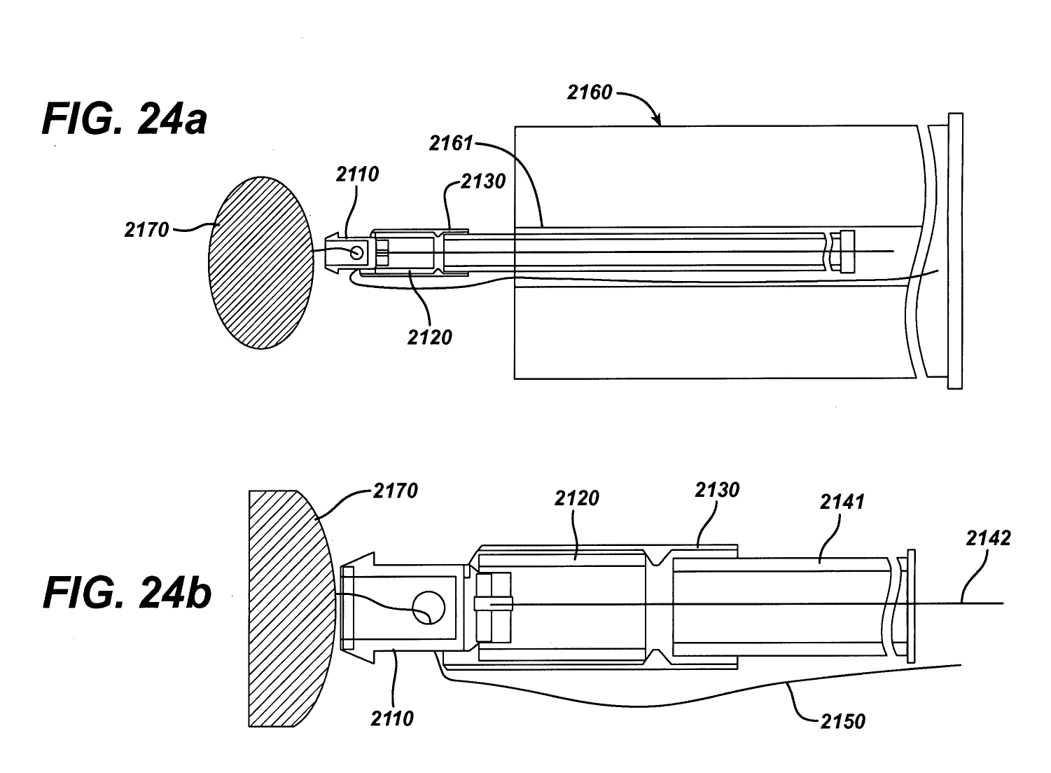

FIG. 23 shows the device in the process of being introduced through the biopsy channel 2161 (more generally the working channel) of a flexible endoscope 2160, whose distal end portion is indicated diagrammatically. Referring to FIGS. 24a and 24b, it can be seen that the suture 2150 is already anchored, for example by a sewing operation, in an area of tissue 2170 within the body of a patient, either human or animal. The manner in which this sewing is effected does not form part of the present invention, and there are various known methods by which such sewing can be effected. However, it is a preferred (and the device of the present invention is designed so that this is possible) that in the subsequent locking and cutting operation the suture 2150 should pass from the tissue, up through the biopsy channel of the endoscope, and thence to the exterior of the patient. This will automatically be the case if the suture was already present in the biopsy channel during the sewing procedure. If this was not the case then the endoscope tube can to be threaded onto the suture 2150 and passed down into the patient, so that its distal end is adjacent the tissue 2170, before the locking and cutting operation begins.

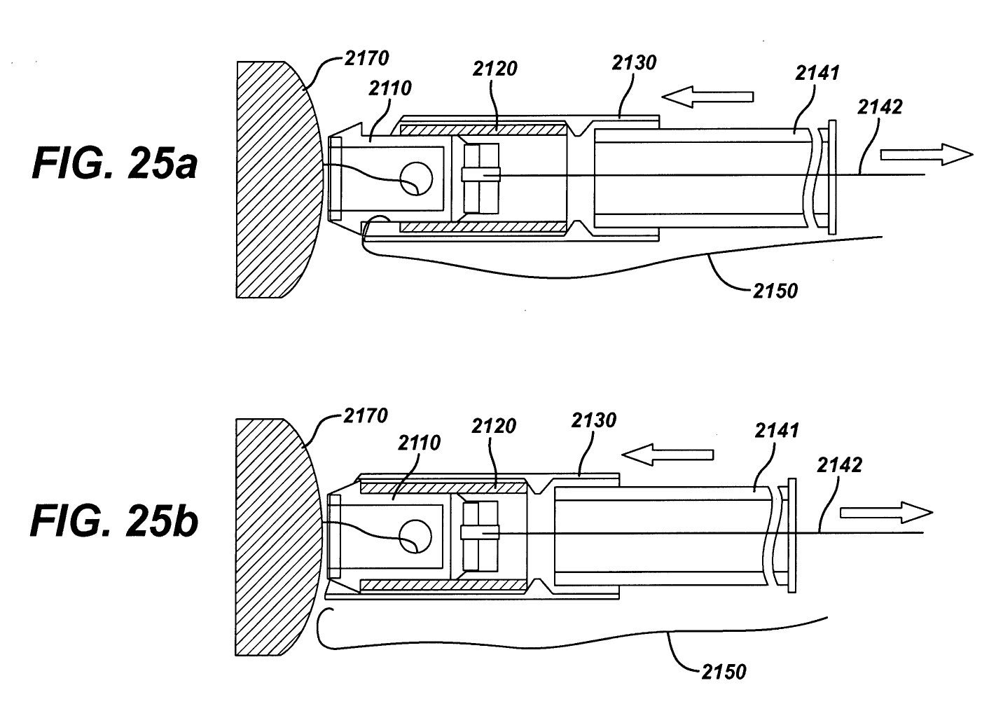

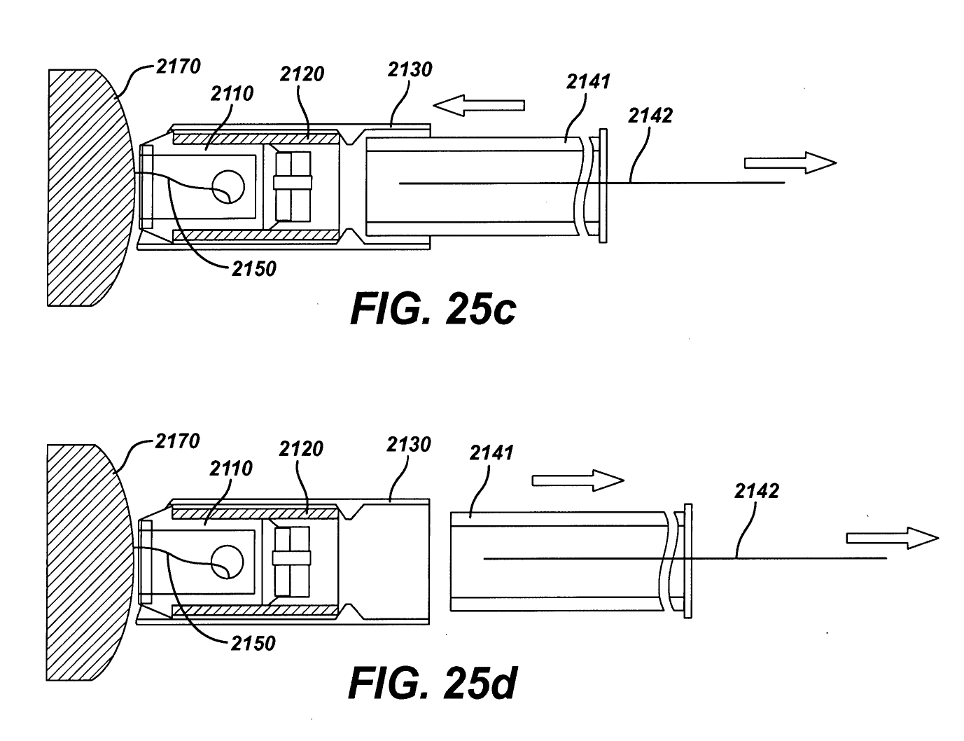

FIGS. 24a and 24b show the device after its head, i.e. the combination of components 2110, 2120 and 2130, has passed through the biopsy channel, and is adjacent the area of tissue 2170. The suture is then locked and cut as described below with reference to FIGS. 25a through 25d. It is noted at this point that in each of these figures the plastic inner tube 2120 is hatched to make it easier to identify it, and, therefore, the other components.

In the first stage of the process, show by FIG. 25a, tension is exerted on the inner wire 2142 of the Bowden cable, and a corresponding compression on the sheath 2141, so that the steel piston 2110 slides partially into the tube 2120. It is able to do this because the plastic tube can deform sufficiently to permit this, even though the initial outer diameter of the distal portion 2114 of the piston 2110 is larger than the inner diameter of the distal portion of the tube 2120. This traps the suture 2150 between the outer wall of the piston 2110 and the inner wall of the tube 2120.

The relative movement of the piston 2110 and tube 2120 is then continued, as shown in FIG. 25b, so that the cutting edge 2112 of the piston 2110 engages the suture 2150 and severs it.

The locking and cutting device is so designed that the transition to the state shown in FIG. 25b can be achieved by applying a relatively low tension to the wire 2142 of the Bowden cable. The tension required is low enough that the connection between the wire portion 2143 and the piston 2110 remains intact. The next step is to apply a higher tension which, as shown in FIG. 25c, causes the wire portion 2143 to break away from its connection to the piston 2110. In the illustrated embodiment this involves the breaking of the connection provided by the solder 2144.

Finally, as shown in FIG. 25d, once this connection has been broken, the continued application of tension to the sheath of the Bowden cable causes it to slide out of the outer tube 2130. Continued pulling on the Bowden cable removes it completely from the patient's body. Once this has been done, all that remains inside the body is what is shown at the left hand side of FIG. 25d, namely the combination of piston 2110 and tubes 2120 and 2130, one inside the other, with the suture 2150 locked between the piston 2110 and the tube 2120. It is to be noted that the cutting edge of the flange 2111 is entirely surrounded by the tube 2130, which avoids any risk of the cutting edge coming into contact with the patient's tissues.

While the present invention has been illustrated by description of various embodiments, it is not the intention of the applicants to restrict or limit the spirit and scope of the appended claims to such detail. Numerous other variations, changes, and substitutions will occur to those skilled in the art without departing from the scope of the invention. Moreover, the structure of each element associated with the present invention can be alternatively described as a means for providing the function performed by the element. It will be understood that the foregoing description is provided by way of example, and that other modifications may occur to those skilled in the art without departing from the scope and spirit of the appended Claims.

What is claimed is:

1. A medical device for locking onto suture comprising:
a. A first endcap, wherein said first endcap includes a hole bored at an oblique angle to a central axis;
b. A second endcap; and
c. An extension spring extending from said first endcap to said second endcap.

2. The medical device of claim 1 wherein first endcap and said second endcap have an outer diameter of about 0.07 inch.

3. The medical device of claim 1 wherein said hole has a diameter of about 0.04 inch, and is bored at an angle of about 45 degrees to the central axis.

4. A medical device for locking onto suture comprising:
a. An endcap; and
b. An extension spring extending from said endcap, said spring having spring coils which, when the spring is in a relaxed state, clamp against said suture.

5. The medical device of claim 4 wherein said endcap has an outer diameter of about 0.07 inch.

6. A medical device for locking onto suture comprising:
a. A distal endcap, wherein said distal endcap includes a hole bored at an oblique angle to the central axis of said endcap;
b. An extension spring extending from said distal endcap;
c. A proximal endcap attached to a proximal end of said spring; and
d. A suture disposed within said hole.

7. A method of using a medical device to lock to suture comprising the steps of:
a. Obtaining a locking device comprising:
i. A first endcap, wherein said first endcap includes a hole bored at an oblique angle to a central axis;
ii. A second endcap;
iii. An extension spring extending from said first endcap to said second endcap;
b. Threading a suture through said hole;
c. Spreading the coils of said extension spring apart; and
d. Closing the coils of said extension spring to pinch said suture within said spring.

8. The method of claim 7 further comprising the step of:
e. Spreading the coils apart to loosen tension in said suture.

9. A medical device for locking onto suture comprising:
a. A body portion, wherein said body portion includes at least one suture channel;
b. A lock sleeve positioned over an outer surface of said body portion, wherein said lock sleeve has an inner surface that closely matches the outer surface of said body portion; and
c. A means for locking said body portion within said lock sleeve.

10. The medical device of claim 9, wherein said locking means comprises a groove in said body portion and a ring projection in said locking sleeve.

11. The medical device of claim 9, wherein there is a clearance of about 0.001 inch or less between the outer surface of said body portion and the inner surface of said locking sleeve.

12. A medical device for locking onto suture comprising:
1) A body portion, wherein said body portion includes at least one suture channel;
2) A lock sleeve positioned over an outer surface of said body portion, wherein said lock sleeve has an inner surface that closely matches the outer surface of said body portion;
3) A means for locking said body portion within said lock sleeve.

13. A medical device for locking onto suture, which comprises an outer tubular member, and an inner tubular member which has a distal portion of a first cross-section and a proximal portion of a second cross-section, the said first portion having an aperture formed therethrough and sized to allow a suture to pass therethrough, the device having a non-locking state in which the said second portion is at least partly received in the outer tubular member, and the said aperture is so located that the suture can pass freely through it, and a locking state in which the said first portion is located at least partially within the outer tubular member, and the suture is locked between the inner and outer tubular members.

14. A device according to claim 13, wherein the first and second portions of the inner tubular member are connected to one another by an intermediate portion.

15. A device according to claim 14, wherein the first and second portions are at least substantially cylindrical, and the intermediate portion is a tapered portion which integrally connects the first and second portions.

16. A device according to claim 13, which comprises pulling means for effecting movement of one of the tubular members relative to the other, the pulling means being connected to the said one member by a connection which is sufficiently strong to enable a force to be applied thereto to effect that relative movement, but which is breakable under a higher force to allow the pulling means to be detached from the tubular members after locking.

17. A medical device for locking onto suture, which comprises a pair of locking members movable with respect to one another from a non-locking position to a locking position, and pulling means for effecting movement of one of the tubular members relative to the other, the pulling means being connected to the said one member by a connection which is sufficiently strong to enable a force to be applied thereto to effect that relative movement, but which is breakable under a higher force to allow the pulling means to be detached from the tubular members after locking.

18. A medical device for locking and cutting a suture, which comprises a first member having an aperture sized to allow a suture to pass through, the first member having a distal end and a proximal end, a second member with respect to which the first member is movably mounted, and means releasably connected to the first member for pulling it in a proximal direction from a first position in which the suture is free to pass through the said aperture, via a second position in which the suture is clamped between the first and second members, to a third position in which the suture is cut by cooperation between the first and second members.

19. A device according to claim 18, wherein the second member is generally cylindrical, and the first member is slidable within the first member, wherein in the first position only the proximal end portion of the first member is received within the second member, in the second position the first member is received within the second member to a greater extent, and in the third position the first member is at least substantially received within the first position, the releasable connection between the pulling means and the first member being arranged to separate when a force is applied to the pulling member sufficiently in excess of that required to move the first member into the third position.

20. A method of using a medical device to lock onto suture comprising the steps:
a. Obtaining a locking device comprising:
1) A body portion, wherein said body portion includes at least one suture channel;
2) A lock sleeve positioned over an outer surface of said body portion, wherein said lock sleeve has an inner surface that closely matches the outer surface of said body portion;
3) A means for locking said body portion within said lock sleeve;
b. Threading a suture through said suture channel;
c. Pushing said locking sleeve over said body portion to trap said suture in a tortuous path; and
d. Locking said locking sleeve to said body portion.

21. A medical device for locking onto suture comprising:
a. A tubular sleeve including an inner channel;
b. A flap disposed within said inner channel of said tubular sleeve; and
c. A detent, wherein said detent is positioned within said inner channel such that said flap is biased to press against said detent.

22. A medical device for locking onto suture comprising:
a. A first gripping surface including a plurality of projections;
b. A second gripping surface, wherein said second grip surface is not in contact with said first grip surface in a default state; and
c. A clasp, wherein said clasp holds said first gripping surface against said second gripping surface in a locked state.

23. A device for cutting a surgical suture, the device comprising a tubular member having a longitudinal axis and a tubular wall with a pair of apertures extending therethrough, the apertures being sized and arranged to permit a surgical suture to pass into the tubular member through one of the pair of apertures and out of the tubular member through the other, a cutting member received within the tubular member, and means for causing longitudinal movement of the cutting member and tubular member with respect to one another in a direction to cause the cutting member to pass at least one of the pair of apertures to sever the suture passing therethrough.

24. A device according to claim 23, wherein the pair of apertures are longitudinally spaced from one another.

25. A device according to claim 24, wherein the pair of apertures are angularly offset with respect to one another about the longitudinal axis of the tubular member.

26. A device according to claim 25, wherein the angular offset of the apertures from one another is 180 degrees, or approximately 180 degrees.
